(19) United States
(12) Patent Application Publication
(10) Pub. No.: US 2005/0136141 A1
(43) Pub. Date: Jun. 23, 2005

(54) Compositions of and derived from strawberry and raspberry and therapeutic uses thereof

(76) Inventors: Gary D. Stoner, Columbus, OH (US); John M. Cassady, Columbus, OH (US)

Correspondence Address: Jerry K. Mueller, Jr.;Mueller and Smith, LPA, 7700 Rivers Edge Drive, Columbus, OH, 43235, US

(21) Appl. No.: 10/951,413

(22) Filed: Sep. 28, 2004

(30) Foreign Application Priority Data
Feb. 28, 2003 (WO) PCT/US03/06279

(51) Int. Cl. A61K 31/525; A61K 31/355; A61K 35/78; A61K 31/015

(52) U.S. Cl. 424/765; 514/474; 514/458; 514/456; 514/251; 514/725; 514/763

(57) ABSTRACT

Isolated berry extracts and components thereof, in a form suitable for use as a foodstuff, dietary supplement, or pharmaceutical composition, are disclosed. The isolated berry extracts or compositions comprising one or a combination of components derived from the berry extracts, can be used as agents for inhibiting a variety of diseases or disorders, for example, cancer or cardiovascular disease. In addition, the invention features novel methods of preparing berry extracts in a form suitable for adding to foodstuffs, dietary supplements, or pharmaceutical.

### RELATED APPLICATIONS

This application claims priority to U.S. Provisional Application No. 60/360,783 filed on Mar. 1, 2002, U.S. Provisional Application No. 60/369,160 filed on Mar. 29, 2002, and U.S. Provisional Application No. 60/425,829 filed on Nov. 12, 2002, the contents of which are incorporated herein by reference.

### GOVERNMENT SPONSORED RESEARCH

This work was supported, in part, by grants from the National Cancer Institute (CA 46535), National Institute of Environmental Health Sciences (5P30 ES00260-40), and National Institute of Dental and Craniofacial Research (PO1 DE12704).

### BACKGROUND OF THE INVENTION

For thousands of years, humankind has relied on plant derivatives for the prevention and treatment of a wide variety of ailments. For example, in China, various teas have been used as a crude medicine for over 4,000 years. And more recently, there has been considerable interest in taking advantage of various plant extracts as a source of health promoting substances such as natural oxidants, phenolic compounds, flavonoids, tocochromanols, and beneficial fatty acids. In part, this trend is due to a growing body of evidence demonstrating that some of these compounds have beneficial properties that may be advantageous in preventing or delaying the onset of disease. Indeed, several epidemiological studies considering the effect of diet on disease such as, e.g., cancer and cardiovascular disease (e.g., because of high cholesterol), have provided leads in the search for naturally-occurring anti-cancer or anti-cholesterol agents. For example, some studies suggest that plant-based diets, rich in whole grains, legumes, fruits and vegetables, may reduce the risk of various types of cancer (Steinmetz et al., Cancer Causes Control. 2:325-357 (1991); World Heath Report 2000, World Health Organization, Geneva, Switzerland (2000).

Similarly, other studies report that populations consuming large amounts of fruit and vegetables have a lower incidence of cardiovascular disease and reduced risk of several types of cancer. Such studies have attributed the beneficial properties of diets rich in fruits and vegetables to the presence of naturally occurring compounds, including various vitamins and minerals, and these compounds have been found in a wide variety of plant sources (Rijnkels et al., Cancer Lett., 114:297-298 (1997); Narisawa et a., P.S.E.B.M. 224:116-122 (2000); Miyagi et al., Nutr. Cancer, 36:224-229 (2000); Reddy et al., Carcinogenesis, 2:21-25 (1981); Kawamori et al., Cancer Res. 59:597-601 (1999); Levi et al., Cancer, 36:2115-2119 (2000); Wang et al., Cancer Lett., 98:63-69 (1995); Kim et al., Chemoprevention Rev., 54:259-279 (1996) and; Quereshi et al., Am. J. Clin. Nutr., 53:1021 S-6S (1991)).

Moreover, additional studies suggest that fruit products are a source of a number of health promoting phytochemicals (Johns et al., Recent Advances in Phytochemistry, pp.31-52, Plenum Press (1997)).

Given that cancer and cardiovascular disease (e.g., cholesterol-related diseases) are two of the major causes of death in the United States, additional research on and identification of fruit-derived therapeutic compounds which, for example, are useful in treating or preventing such diseases, would be of great benefit.

### SUMMARY OF THE INVENTION

The present invention provides novel compounds and therapeutic compositions (e.g., formulations) derived from fruits, in particular berries, and more particularly strawberry and raspberry, as well as novel uses for the compounds and compositions. In particular embodiments, the compounds are formulated as a pharmaceutical, a foodstuff (e.g., added to a foodstuff to enhance its nutritional and/or medical value), or a dietary supplement. In all cases, the compounds and compositions contain, or are enriched for, health promoting components (e.g., antioxidants, vitamin A, vitamin E (tocochromonals), vitamin C (ascorbic acid), folic acid, carotenoids, phenolic compounds, phytosterols, and minerals) that are useful in treating or preventing a variety of health-related disorders and diseases. In addition, the invention provides methods of efficiently producing berry, e.g., strawberry and raspberry extracts (and fractions thereof) enriched for antioxidant activity (and other desirable components) such that the extracts (or fractions) can be added to foodstuffs or used as a dietary supplement or a pharmaceutical composition.

Accordingly, in another embodiment, the present invention provides a method for treating or preventing a disease in a subject, particularly a malignancy (e.g., a cancer), by administering to the subject (e.g., orally or, when appropriate, by other routes) a therapeutically-effective amount of a compound or composition (e.g., an extract or extract fraction) of the invention. The malignancy can be, for example, metastatic, an aerodigestive tract cancer, or a metastatic aerodigestive tract cancer (e.g., an oral, pharyngeal, laryngeal, esophageal, stomach, or colon cancer). In another embodiment, the present invention provides a method for treating or preventing other diseases or disorders associated with oxidative damage such as skin cancer, cardiovascular disease (e.g., due to high cholesterol, i.e., hypercholesterolemia), neurodegenerative disease (e.g., stroke), immunological diseases or conditions, inflammatory diseases or conditions such as arthritis, dermatological conditions, and opthalmological conditions, in a subject, by administering to the subject a therapeutically-effective amount of a compound or composition of the invention (e.g., an extract or extract fraction of the invention). The compounds or compositions of the invention, when administered to a subject, may also be used to retard aging. The factors contributing to aging being, for example, oxidative mechanisms and compounds, for example, free radicals, which can damage cellular lipids, proteins, and genetic material.

Novel compositions of the invention are derived (e.g., isolated) from, or contain components of strawberry and raspberry fruits, for example, strawberry and black raspberry, and combinations thereof. Particular compositions identified by way of the present invention as having significant therapeutic value include and/or are derived from strawberry and/or raspberry (e.g., black raspberry) fruits which have been, for example, pureed, freeze-dried (referred to as a berry extract), organically extracted (e.g., by solvent extraction of a berry extract, thereby resulting in a berry extract fraction), and combinations thereof. Such berry extracts and fractions thereof, can then be formulated in a variety of manners, such as a dietary supplement, a pharmaceutical, or as an additive to a foodstuff. They may also contain additional desirable compounds such as carbohydrates, some proteins, fiber, and combinations thereof.

In a related embodiment, the present invention further provides therapeutic compositions containing novel combinations and/or ratios of health-promoting compounds derived (e.g., isolated) from strawberry and raspberry (e.g., black raspberry). Such compounds can be isolated from, for example, strawberry and raspberry (e.g., black raspberry), extracts and/or fractions. By way of non-limiting illustration, such compounds can include antioxidants, vitamins (e.g., vitamin A, vitamin E (tocochromonals), vitamin C (ascorbic acid), folic acid, carotenoids, phenolic compounds, phytosterols, minerals, or combinations thereof.

In another aspect, the invention provides a method for isolating berry extracts, and optionally, fractions thereof, so that the extracts and/or fractions can be administered to a patient or to an animal as a therapeutic agent. In one embodiment, the method involves freeze drying the berries, followed by pulverization into a powder, then exposing the resultant extract to low temperature, and removing an amount of water content, e.g., under a vacuum (e.g., about half an atmosphere, e.g., 380 millitorr, e.g., by sublimation), thereby resulting in a freeze-dried extract enriched for antioxidant activity and other beneficial compounds. In a related embodiment, the berry extract is then exposed to an organic solvent to produce an extract/solvent mixture, and the solvent portion of the extract/solvent mixture is then removed, thereby producing an isolated berry extract fraction substantially free of solvent, e.g., greater than 90% free of solvent, preferably, greater than 99% free of solvent. When the solvent is well tolerated by an animal, e.g., ethanol, the solvent concentration can remain as high as appropriate to deliver the beneficial components to the animal (e.g., fractions in 50% ethanol). Other solvents include dichloromethane, methanol, ethanol, acetone, and combinations thereof, with preferred combinations being about a 1:1 combination of dichloromethane and methanol, about a 1:1 combination of dichloromethane and ethanol, about a 1:1 combination of acetone and methanol, or about a 1:1 combination of acetone and ethanol. Fractions derived from an extract (e.g., a freeze-dried extract) preferably represent at least about 50 to 55% of the starting extract material.

In a related embodiment, the berry extract or extract fraction of the above method is enriched, by about 1-5 fold, preferably 5-10 fold, more preferably by about 10 fold or greater, for antioxidant activity and the presence of, e.g., one or more of the following: a vitamin (e.g., vitamin A, vitamin E, vitamin C, folic acid), carotenoid (e.g., α-carotene, β-carotene, zeaxanthin, and lutein), a phenolic compound (e.g., ellagic acid, ferulic acid, anthocyanin, cyanidin, quercetin, pelargonidin, kaempferol, and analogs thereof), a phytosterol (e.g., β-sitosterol, campesterol, stigmasterol, and analogs thereof), and a mineral (e.g., calcium, magnesium, potassium, zinc, and selenium).

In another embodiment, the extracts of the methods (or fractions thereof) enriched for, e.g., antioxidant activity, are suitable for use in a foodstuff, a dietary supplement, or a pharmaceutical composition. Accordingly, the extracts of the invention (or fractions thereof) can be used in the treatment of a subject in need of an antioxidant therapy or having an antioxidant responsive disease or condition, such that treatment is achieved.

Other features and advantages of the invention will be apparent from the following detailed description and claims.

### BRIEF DESCRIPTION OF THE DRAWINGS

FIG. 1 shows a flow chart of a method for extracting desirable fractions from freeze-dried berries. Fractions containing desirable compounds that can be further partitioned are also shown. Abbreviations: RU=black raspberry; FA=strawberry.

FIG. 2 shows a flow chart of a method for extracting desirable fractions from freeze-dried berries. Chromatographic steps and alternative fractionation approaches are also shown. Abbreviations: RU=black raspberry; FA=strawberry.

FIG. 3 shows the inhibition of oral cancer in the hamster cheek pouch (HCP) in hamsters fed diets containing 5% and 10% black raspberry extracts as compared to controls.

FIG. 4 shows the inhibition of BPDE induced activator protein-1 (AP-1) activity in mouse epidermal cells (i.e., JB-6 clone 41) transfected with AP-1 (P⁺1-1 cells) treated with black raspberry extract fractions as compared to controls.

FIG. 5 shows the dose-dependent inhibition of BPDE induced AP-1 activity in cells (P⁺1-1 cells) treated with the methanol extract fraction of black raspberries.

FIG. 6 shows the inhibition of BPDE induced NFκB (b) activity in cells (mass1 cells) treated with black raspberry extract fraction (RU-ME).

FIG. 7 shows the dose-dependent inhibition of BPDE induced NFκB activity in cells (mass1 cells) treated with the methanol extract fraction of black raspberries.

FIG. 8 shows the inhibition of BPDE induced activation of MAPKs and IκBα phosphorylation and degradation in mouse epidermal cells (JB-6 clone 41) treated with black raspberry extract fraction (RU-ME) as compared to controls as determined by immunoblot.

FIG. 9 shows that the inhibitory activity of the black raspberry extract fractions is independent of BPDE induced p53-dependent transcription activity in mouse epidermal cells (JB-6 clone 41) transfected with p53 (mass1 cells).

FIG. 10 shows a chromatogram obtained upon HPLC analysis of the methanol fraction of lyophilized black raspberry extracts.

FIG. 11 shows the chemical structure of several active compounds identified in the lyophilized black raspberry extracts of the invention, i.e., cyanidin, quercetin, pelargonidin, and kaempferol.

FIGS. 12-14 show LC-ESI-MS chromatograms obtained upon analysis of the methanol fraction of lyophilized black raspberries for the presence of cyanidin, quercetin, pelargonidin, and kaempferol, and sugar conjugates thereof.

### DETAILED DESCRIPTION OF THE INVENTION

In order to provide a clear and consistent understanding of the specification and claims, including the scope to be given such terms, the following definitions are provided.

### Definitions

The term “analog” as in “a compound or analog thereof”, is intended to include compounds that are structurally similar but not identical to the compound, but retain some or all of the anti-cancer properties of the compound.

As used herein the term “anti-cancer activity” or “anti-cancer properties” refers to the inhibition (in part or in whole) or prevention of a cancer as defined herein. Anti-cancer activity includes, e.g., the ability to reduce, prevent, or repair genetic damage, modulate undesired cell proliferation, modulate misregulated cell death, or modulate mechanisms of metastasis (e.g., ability to migrate).

The term “anti-hypercholesterolemic activity” and “cholesterol lowering activity” refers to the ability to regulate cholesterol metabolism or reduce serum cholesterol levels in a subject.

The term “antioxidants” includes chemical compounds that can absorb an oxygen radical, e.g., ascorbic acid and phenolic compounds.

The term “antioxidant activity” refers to a measurable level of oxygen radical scavenging activity, e.g., the oxygen radical absorbance capacity (ORAC) of an extract, fraction, or compound.

The term “antioxidant responsive condition” includes any disease or condition that is associated with the presence of undesired oxidation, oxygen radicals, or other free radicals.

The term “berry” is intended to mean a fruit with external seeds such as a strawberry (e.g., strawberries of the genus Fragaria, e.g., Fragaria ananassa) and a fruit containing clustered berries (each with a seed) such as a raspberry (e.g., a red or black raspberry, e.g., raspberries of the genus Rubus, e.g., Rubus occidentalis).

The term “berry extract” includes a berry extract isolated from its natural context (i.e., the fruit), e.g., concentrated freeze-dried berries (e.g., lyophilized). Preferably, “isolated berry extract” of the invention is enriched for the presence of increased antioxidant activity, for example, has a high oxygen radical absorbance capacity (ORAC) (e.g., a value at least about 5.0 per mg, and preferably, between at least about 5-10, more preferably, between at least about 10-15, most preferably, at least about 15 or greater), has increased levels of antioxidants, has a high vitamin content (e.g., vitamin A, vitamin E (tocochromonal) content, vitamin C (ascorbic acid), folic acid, other desirable components (e.g., carotenoids, phenolic compounds, phytosterols, and minerals), and is substantially free of undesired impurities, e.g., stems.

The term “berry extract fraction” includes a berry extract that has been fractionated with a solvent and is, preferably substantially free of solvent (e.g., at least 80-90%, preferably 90-99%, more preferably greater than 99%, and most preferably greater than 99.7%) as determined by standard techniques (e.g., gas chromatography), and/or off-flavors (as determined by taste and smell).

The term “cancer” or “malignancy” are used interchangeably and include any neoplasm (e.g., benign or malignant), such as a carcinoma (i.e., usually derived from epithelial cells, e.g., aerodigestive tract cancer, such as an oral, esophageal, or colon cancer) or sarcoma (usually derived from connective tissue cells, e.g., a bone or muscle cancer) or a cancer of the blood, such as a erythroleukemia (a red blood cell cancer) or leukemia (a white blood cell cancer). A “malignant” cancer (i.e., a malignancy) can also be metastatic, i.e., have acquired the ability to transfer from one organ or tissue to another not directly connected, e.g., through the blood stream or lymphatics.

The term “cardiovascular disease” includes, for example, hypercholesterolemia, thrombotic disease, and artherogenic disease.

The term “carotenoid” includes, for example, α-carotene, β-carotene, zeaxanthin, and leutin.

The term “dietary supplement” includes a compound or composition used to supplement the diet of an animal or human.

The term “exogenous” means the component is derived or obtained from a source other than berries. Exogenous compounds suitable for adding to a berry extract of the invention (or fractions thereof) include, for example, one or more pharmaceuticals, chemotherapeutic agents, and/or radiotherapy.

The term “foodstuff” includes any edible substance that can be used as or in food for an animal or human. Foodstuffs also include substances that may be used in the preparation of foods such as cooking oils or food additives. Foodstuffs also include dietary supplements designed to, e.g., supplement the diet of an animal or human.

The terms “health promoting”, “therapeutic” and “therapeutically effective” are used interchangeably herein, and refer to the prevention or treatment of a disease or condition in a human or other animal, or to the maintenance of good health in a human or other animal, resulting from the administration of a berry extract (or fraction thereof) of the invention, or a composition derived therefrom. Such health benefits can include, for example, nutritional, physiological, mental, and neurological health benefits.

The term “hypercholesterolemia” refers to abnormally high serum levels of cholesterol, typically due to defective cholesterol metabolism in a subject or diet.

The term “isolated” refers to the removal or change of a composition or compound from its natural context, e.g., the berry.

The term “mineral” includes, e.g., any mineral that is naturally present at some measurable level in the berry extracts (or fractions thereof) and includes, e.g., calcium, magnesium, potassium, zinc, and selenium.

The term “native” refers to the originating berry source.

The term “pharmaceutical composition” or “therapeutic composition” refers to a composition formulated for therapeutic use and may further comprise, e.g., a pharmaceutically acceptable carrier.

The term “pharmaceutically effective amount” refers to an amount effective to achieve a desired therapeutic effect, such as lowering tumor incidence, metastasis, undesired lipid levels in the blood, preventing thrombosis, preventing or treating inflammatory diseases, immunoregulatory diseases, fever, edema, cancer, or signs of aging.

The term “phenolic compound” includes a compound that has an aromatic acid having one or more hydroxyl groups on the benzene ring and is naturally present at some measurable level in the berry extract (or fraction thereof) and includes, for example, ellagic acid, ferulic acid, anthocyanin, cyanidin, quercetin, pelargonidin, kaempferol, and analogs thereof.

The term “physically disrupting” includes any appropriate physical manipulation (e.g., by mechanical means, e.g., using a masher, juicer, pulper, or, e.g., by sonication) that breaks (e.g., decharacterizes) the fruit into, e.g., skin, seeds and juice, e.g., into a puree.

The term “phytosterol” includes any sterol e.g., that is naturally present at some measurable level in the berry extracts (or fractions thereof) and includes, for example, _-sitosterol, campesterol, stigmasterol, and analogs thereof.

The term “vitamin” includes, for example, vitamin A, vitamin E, vitamin C, folic acid, but also any other art recognized vitamins.

The term “vitamin A” generally includes retinal, retinol, retinoic acid, or a combination thereof.

The term “vitamin C” generally refers to ascorbic acid.

The term “vitamin E” generally includes tocochromanol compounds such as tocopherol or tocotrienol compounds.

### Overview

The present invention is based on the identification of therapeutic berry extracts, for example, strawberry and black raspberry extracts, and fractions or compounds isolated from the extracts (e.g., a berry extract fraction), having novel therapeutic and/or health promoting value. In particular, therapeutic berry extracts of the invention (and fractions thereof) are shown herein to exhibit significant anti-cancer and anti-hypercholesterolemia activity when administered to a subject in vivo and when tested in vitro.

In a particular embodiment, therapeutic methods of the invention employ physically disrupted berry fruit, preferably a puree free of cap stems, which is freeze-dried to produce a berry extract substantially free of water content and is enriched for a number of health promoting compounds and exhibits e.g., significant anti-cancer properties when administered to a subject. Moreover, a variety of particular health promoting compounds derived from prepared berry have been identified and are discussed below.

In another embodiment, therapeutic methods of the invention employ berry products (e.g., extracts, or fractions thereof) which are novel sources of compounds having significant therapeutic value, in, for example, the prevention or treatment of cancer, particularly, aerodigestive cancers. In addition, as described herein, a subset of these berry derivatives are also enriched with compounds suitable for treating cardiovascular disease related to, for example, high cholesterol (hypercholesterolemia).

In another embodiment, therapeutic methods of the invention employ compounds derived from berry extracts which, as shown herein, have anticancer activity (e.g., reduced metastasis rates) e.g., when prepared in concentrated form and administered to a mammal in vivo.

Accordingly, the identification of particular beneficial compounds in berry extracts and derivatives thereof has allowed for the development of convenient methods and compositions (e.g., formulations) for administering therapeutic compounds to treat or prevent particular diseases. Moreover, the therapeutic compounds and compositions described herein have the additional advantage of being readily manufactured into palatable forms (e.g., as foodstuffs such as juices and food bars or as dietary supplements) for convenient oral administration.

Methods for obtaining and preparing the berry extracts of the invention, identifying (e.g., characterizing) and obtaining therapeutic components of the products, evaluating biological activity in vitro and in vivo of the products and components, and methods of using the products and novel compositions containing the products or combinations of components isolated from the products, are discussed in the following subsections.

### Methods for Preparing Berry Extracts and Fractions Thereof

Berry extracts of the invention (and fractions thereof) may be isolated from whole berry, preferably freshly harvested berries, using any suitable art recognized method. Preferred derivatives include berry extract, or fractions thereof, that optionally, have been freeze-dried. The berries may be freeze-dried using any art recognized method. In a particular method, the berries are freeze-dried by first physically disrupting the berries resulting in a puree which is then further processed to be substantially free of impurities or undesired solids, e.g., stems. The puree is then poured into a shallow vessel and quickly exposed to low temperature, i.e., flash frozen, for example at −20° C. or lower, preferably under a vacuum for removal of water content (lyophilization). The resultant berry extract, as compared to the native fruit by weight, is typically enriched for, e.g., antioxidant activity, antioxidant compounds, and other compounds described herein, by a factor of at least about 1-5 (i.e., ˜100-500%), preferably, by a factor of at least about 5-10 (i.e., ˜500-1000%), more preferably by a factor of at least about 10 or more (i.e., ˜1000% or more).

The resultant extract (i.e., lyophilized) may be, optionally, fractionated by adding an organic solvent to produce an extract/solvent mixture, and removing the solvent portion of the extract/solvent mixture such that an isolated berry extract substantially free of solvent results. By selection of particular solvents, as described below, fractions enriched for particular compounds with health promoting activities, can be obtained. In one embodiment of the extraction method, an isolated berry extract results that is suitable for use in a foodstuff, dietary supplement, or pharmaceutical composition.

In all cases, the berry extracts or fractions are preferably obtained in a form suitable for use in a foodstuff, dietary supplement, or pharmaceutical composition. Further, it is understood that with regard to any of the techniques for preparing a berry extract or derivative described herein, it may also be desirable to avoid exposing the derivative, or component thereof, to oxygen by, e.g., protective blanketing of the derivative or component with an inert gas (e.g., carbon dioxide or nitrogen gas), or by, e.g., exposing the derivative or component, where appropriate, to low temperature, a stabilizer, or a combination of these conditions.

### Berry Extracts and Fractions Thereof

As part of the present invention, several berry extracts, including strawberry and black raspberry (including fractions thereof) were analyzed for health promoting antioxidant activity and various compounds. These berry extracts were analyzed using both chemical analysis and bioactivity assays as described herein. In addition, a number of berry extract fractions were also studied for their in vitro and in vivo therapeutic activity and analyzed for health promoting compounds (see Tables 1-3).

Accordingly, by way of the studies described herein, it was shown that particular berry extracts are novel sources of therapeutically beneficial antioxidant activity (e.g., as measured by the oxygen radical absorbance capacity (ORAC) of the extracts) as well as compounds such as a vitamin A, vitamin E (tocochromanols), vitamin C (ascorbic acid), folic acid, carotenoids, phenolic compounds, phytosterols, minerals, and combinations thereof. The berry extracts prepared as described herein provide several advantages over currently known sources of such therapeutically beneficial compounds including, for example, remarkably high levels of antioxidant activity as well as the presence of many desirable components. Accordingly, the berry extracts of the invention, or components thereof, can be used in foodstuffs, dietary supplements, and pharmaceutical compositions.

Accordingly, in one embodiment, the invention provides a berry extract, for example, a strawberry or black raspberry extract, or a composition comprising one or more components of such an extract, as listed, respectively, in Tables 2 and 3, which promotes health in a human or other animal. The berry extracts or composition derived therefrom are also preferably substantially enriched for antioxidant activity, for example, possess a high value for oxygen radical absorbance capacity (ORAC) as shown in Table 1. The berry extracts or composition derived therefrom also can contain one or more exogenous (i.e., externally added) compounds to further enhance the therapeutic value of the berry extracts or composition derived therefrom, for example, by acting in synergism with one or more native components of the berry extract.

The strawberry and black raspberry extracts of the invention can contain one or more of the following compounds: vitamins (e.g., vitamin A, vitamin E, vitamin C, and folic acid); carotenoids (e.g., _-carotene, β-carotene, zeaxanthin, lutein); phenolic compounds (e.g., ellagic acid, ferulic acid, anthocyanin, cyanidin, quercetin, pelargonidin, kaempferol, and analogs thereof); phytosterols (e.g., β-sitosterol, campesterol, and stigmasterol, and analogs thereof); and minerals (e.g., calcium, magnesium, potassium, zinc, and selenium). In addition, exogenous compounds, such as other vitamins (e.g., vitamins underrepresented) and/or chemotherapeutic agents, can be added to the berry extracts of the invention and compositions derived therefrom, to achieve a synergistic effect.

In addition, the berry extracts of the invention contain high levels of antioxidant activity as measured by the oxygen radical absorbance capacity (ORAC) of the extracts. In particular, black raspberry extracts are especially enriched for such antioxidants. Accordingly, the berry extracts have a high antioxidant activity (in addition to other properties discussed herein).

The therapeutic benefit of the antioxidant activity and other compounds of the extracts is summarized under the following subsections.

### Antioxidant Activity

An important activity found in the berry extracts of the invention is antioxidant activity, e.g., as determined by the oxygen radical absorbance capacity (ORAC) value found for each extract. Thus, the berry extracts of the invention (and fractions thereof) have the advantage of being potent delivery systems for antioxidants. Antioxidants, as discussed below, include, e.g., vitamin E, vitamin C, and phenolic compounds.

### Vitamin A

The berry extracts of the invention also contain vitamin A which generally includes any member (or combination thereof) of a family of fat-soluble vitamins such as retinol, retinal, and retinoic acid. These compounds play an important role in vision, bone growth, reproduction, cell division and differentiation, immunoregulation, and lowering cancer risk.

### Vitamin E (Tocochromanols)

The berry extracts of the invention also contain vitamin E which generally comprises tocochromanols (a class of compounds that includes tocopherols and tocotrienols). A large body of research has shown the importance of tocopherols and tocotrienols in the defense against numerous biological disorders.

Accordingly, the berry extracts of the invention and compositions derived therefrom (e.g., fractions rich in vitamin E) can be used to treat respiratory, inflammatory, neurological, dermatological, opthalmological, and gastroenterological diseases. Surprisingly, the amount of vitamin E (tocochromanols) determined to be in the berry extracts of the invention is present at high levels in both strawberry and black raspberry extracts (respectively, 5-6 mg/100 gm; ˜11 mg/100 gm).

The use of vitamin E as an anticarcinogenic agent has been recognized for a number of years (Haenszel et al., Int. J. Cancer, 36:43-48 (1985); Menkes et al., N. Engl. J. Med., 315:1250-1204 (1986); Stahelin et al., Ann. NY Acad. Sci., 570:391-399 (1989)). In addition, in vitro and in vivo studies, including human studies, have demonstrated that vitamin E interferes with the development of carcinogenesis that results from exposure to various environmental factors known to enhance oxidant stress (Borek et al., In, Mechanisms of cellular transformation by carcinogenic agents, New York, Pergamon (1987), Borek et al., In, Medical, biochemical and chemical aspects of free radicals, Amsterdam, Elsevier, (1989); Borek et al., Proc. Natl. Acad. Sci. USA 83:1490-1494 (1986); Proc. Natl. Acad. Sci. USA, 88:1953-1957 (1991)). (Ames et al., Science 230:271-279 (1987); Doll et al., J. Natl. Cancer Inst. 66:1193-1194 (1981): Greenwald et al., Cancer 65:1483-1490 (1990); Menzel et al., J. Agr. Food Chem., 20:481-486 (1972)).

### Vitamin C

The berry extracts of the invention also contain vitamin C (ascorbic acid) which can function as an antioxidant. Vitamin C is also useful for promoting healthy teeth and gums, absorption of iron, maintenance of connective tissue and the immune system.

### Folic Acid

Berry extracts of the invention also contain measurable levels of folic acid which acts a coenzyme (with other vitamins (vitamins B-12 and vitamin C) in the metabolism of proteins and in the synthesis of new proteins) and is necessary for the production of red blood cells and the synthesis of DNA, tissue growth and cell function. Adequate levels of folic acid are required to prevent neural tube defects during human embryogenesis.

### Carotenoids

Berry extracts of the invention also contain measurable levels of carotenoids. Typical carotenoids found within the berry extracts of the invention include α-carotene, β-carotene, zeaxanthin, and lutein. The health promoting effects of the carotenoids of the invention include reducing the risk of developing several kinds of cancer, including stomach, colorectal, esophagus, larynx, and lung cancer.

### Phenolic Compounds

Berry extracts of the invention also can contain one or more phenolic compounds, such as ellagic acid, ferulic acid, and anthocyanins (but also, e.g., hydrobenzoic acid, hydroxycinnamic acid, flavonoids (e.g., cyanidin, quercetin, pelargonidin, kaempferol, and analogs thereof), flavanols, flavan-3-ols, and/or tannins). Such phenolic compounds can act as potent antioxidants and, therefore, can prevent or delay oxidation reactions which cause various diseases.

Accordingly, the berry extracts of the invention and compositions derived therefrom (e.g., certain extract fractions) can be used as antioxidants. For example, they can inhibit lipid peroxidation, scavenge free radicals and active oxygen, inactivate lipoxygenase, and chelate iron ions. Moreover, epidemiological studies have demonstrated that the consumption of phenolic compounds is associated with a reduced risk of cancer. Accordingly, the berry extracts of the invention and compositions derived therefrom (e.g., fractions rich in phenolic compounds) can be used to prevent cancer with few side effects.

In particular, the black raspberry and strawberry extracts of the invention contain significant quantities of various polyphenols including ellagic acid, ferulic acid, and multiple anthocyanins. Ellagic acid alone has demonstrated inhibitory effects against skin, lung, liver, esophagus and colon cancer in animals. In addition, ellagic acid activates Hageman factor (involved in blood clotting); inhibits replication of certain DNA viruses such as adenovirus and herpesvirus; inhibits enzymes involved in the synthesis of HTLV-3 (AIDs) virus; inhibits the bioactivation and stimulates the detoxification of certain chemical carcinogens; scavenges the ultimate carcinogenic metabolite of benzo(a)pyrene, a ubiquitous environmental carcinogen; exhibits antimutagenic activity in the AMES mutagenesis assay; and, has therapeutic effects against tumors in animals. In addition, ellagic acid is a strong antioxidant. Ferulic acid also exhibits antimutagenic and antioxidant activity. The anthocyanins impart color to berries and many are polyphenols that exhibit antioxidant activity.

### Phytosterols

In particular, the berry extracts of the invention can contain one or more phytosterols (plant sterols), including, but not limited to, _-sitosterol, campesterol, and stigmasterol, and analogs thereof.

Phytosterols have been shown to inhibit the absorption of cholesterol from the intestine, and decrease blood serum cholesterol. It has been proposed that, in the intestine, phytosterols act by reducing the solubility of cholesterol in the lipid and micellar phases with a consequential decrease in cholesterol absorption. Plant sterols are also reported to inhibit colon cancer and breast cancer development.

Accordingly, the berry extracts of the invention and compositions derived therefrom (e.g., fractions rich in phytosterols) can be used, for example, in the treatment of patients with cardiovascular disease or as chemopreventative agents against colon cancer and breast cancer.

### Minerals

Berry extracts of the invention also contain high levels of minerals. Typical minerals found within the berry extracts of the invention include calcium, magnesium, potassium, zinc, and selenium. The health promoting effects of minerals found within the extracts of the invention include, for example, reducing osteoporosis and cancer risk (calcium), maintaining electrolyte balance (magnesium and potassium), maintaining immune system function (zinc), and reducing cancer risk (selenium).

### Methods for Isolating, Identifying, and Analyzing Specific Components from Berry Extracts

To isolate and analyze constituent therapeutic components (compounds) from the berry extracts of the invention, a variety of art-recognized techniques and assays can be employed. For example, phenolic compounds of the strawberry and raspberry extracts and derivatives of the invention can be analyzed and extracted using HPLC analysis and solvent extraction, respectively. The isolated extracts can be dissolved in an organic solvent, for example, methanol (or ethanol, which can be administered to animals, e.g., humans) and then extracted with a methanol/water solution (or ethanol/water) followed by centrifugation. The extract can then be dried, and the residue can be resuspended in methanol/water for HPLC analysis.

Other components of the extracts, for example, carotenoids, phenolic compounds, phytosterols can be extracted and analyzed using, for example, thin layer chromatography and high-performance liquid chromatography. For example, the material can be fractionated on thin-layer chromatography (TLC) plates where the individual bands that are subsequently resolved can be scraped and extracted with a chloroform/methanol solvent. These resultant samples can then be analyzed using, e.g., gas and high-performance liquid chromatography (HPLC).

Such isolated components, which can be separated as “value added” fractions (e.g., fractions having therapeutic value), are typically rich in at least one beneficial component identified from the berry extracts or factions thereof described herein. These isolated components or fractions may be further combined to provide a composition rich in more than one component or, e.g., a desired combinations thereof.

In addition, a particular formulation intended for the treatment or prevention of a particular disease or condition may be formulated to be rich in those components having a therapeutic effect on the disease or condition (e.g., associated with affecting a change in any of the mechanisms associated with that particular disease or condition). For example, a formulation suitable for administering to a subject with cancer is preferably rich in berry extract-derived components having antioxidant activity and other anti-cancer properties, whereas a formulation for administering to a subject with cardiovascular disease (e.g., hypercholesterolemia) is preferably rich in phytosterols. A subject with a dietary need, may be administered a formulation rich in, for example, beneficial vitamins or minerals.

### Methods for Evaluating Therapeutic Properties of Berry Extracts And Components Derived Therefrom

In another embodiment, the strawberry or raspberry extracts of the invention, and compositions derived therefrom, can be tested for their in vivo therapeutic effect by administering (e.g., orally) the extracts or compositions in a suitable form (e.g., as a food stuff, dietary supplement, or pharmaceutical composition) to a human or other animal, and then observing the physiological effect (e.g., compared to a control). The human or animal can be, for example, suffering from a disease or condition, such as those described herein (e.g., cancer or hypercholesterolemia). Thus, a reduction in the physical symptoms of the disease can be measured as an indication of the therapeutic efficacy of the strawberry or raspberry extracts or compositions derived therefrom.

In another approach for evaluating anti-tumor activity, strawberry or raspberry extracts of the invention or compositions derived therefrom (e.g., a fraction thereof) can be used in a controlled animal study where tumors are induced in the animal via diet (or by other appropriate routes such as injection, e.g., by intraperitoneal, subcutaneous, or intravenous injection), by applying a chemical tumor promoter to the skin, or by the implantation of tumor cells in the presence or absence of the test agent. Various assays, such as those described below, can then be used to examine the progression of carcinogenesis in the presence or absence of the administration of the extracts or compositions of the invention.

The health promoting properties of berry extracts of the invention and compositions derived therefrom also can be evaluated using a variety of art-recognized cell-based assays. For example, the antioxidant effects on cells caused by exposure to a berry extract of the invention or a composition derived therefrom can be determined by an oxygen radical absorbance capacity (ORAC) assay or electron spin resonance technology as described herein. Typically, the extracts of the invention have enriched antioxidant activity as measured by either of these technologies.

The extracts of invention were also found to be non-toxic even at high doses.

### Methods of Use

Treatment of Cancer

In one embodiment, a berry extract of the invention and compositions derived therefrom (particularly those having antioxidant activity) can be administered to a human or other animal to treat or prevent a variety of cancers. In particular, the extracts of the invention are especially well-suited for inhibiting the development of cancers of the aerodigestive tract in animals and humans such as oral, laryngeal, pharyngeal, esophageal (squamous cell carcinoma and adenocarcinoma), stomach, and colon cancer. Other disease indications include preneoplastic lesions in humans such as epithelial dysplasia of the esophagus, development of Barrett's esophagus, oral leukoplakia and erythroplakia, and colonic polyps. The extract and compositions derived therefrom also can be administered in combination with other anti-cancer agents. In particular, the berry extracts of the invention and compositions derived therefrom can be administered with other nutrients, chemotherapy, and/or radiotherapy for the treatment of, for example, an aerodigestive cancer.

Other chemopreventive agents suitable for coadministration for inhibiting development of tumors, e.g., tumors of the oral cavity, when administered before, during, or after initiation by chemical carcinogens include glutathione, beta-carotene, limonin, retinyl acetate, Ocimum sanctum, diallyl sulfide, vitamin E, protease inhibitors from soybeans, ibuprofen, green coffee beans, green tea polyphenols, curcumin, quercetin, and mint. Of these, beta-carotene, retinyl acetate, Ocimum sanctum, diallyl sulfide, retinoids, protease inhibitors, green tea, curcumin, and similar synthetic compounds are suitable for preventing tumor formation when given post-initiation, i.e., after exposure to a chemical carcinogen.

Treatment of Heart Disease

In another embodiment, berry extracts of the invention and compositions derived therefrom (particularly those having high antioxidant activity) can be used to treat or prevent heart disease. Indeed, the efficacy of vitamin E (tocochromonals) in reducing cholesterol levels in animals, including humans, is well supported in the scientific literature.

Accordingly, the berry extracts of the invention, and compositions derived therefrom, can be used in the treatment of high cholesterol (cholesterolemia) and other associated conditions such as heart disease.

Treatment of Other Diseases and Disorders

In yet another embodiment, the berry extracts of the invention and compositions derived therefrom (particularly those having high antioxidant activity) can be used in the treatment or prevention of a wide range of other diseases and disorders that include, respiratory, inflammatory, neurological, dermatological (e.g., actinic keratosis and dysplastic nevi of the skin, skin cancer), cardiovascular disease, stroke, inflammatory diseases (e.g., arthritis), as well as inhibiting aging. Indeed, a large volume of reported research provides evidence that antioxidants (and other compounds, e.g., vitamin E) play a critical role in the above-mentioned conditions.

Accordingly, the berry extracts of the invention and compositions derived therefrom having both of these properties are especially well suited for the prevention and/or treatment of a broad spectrum of biological conditions. Moreover, such extracts and compositions of the invention also are well suited to the treatment of any yet to be characterized biological disorders or diseases that, at some level, are affected by or controlled by a mechanism associated with these properties.

Hypercholesterolemic diseases and conditions that can be treated using the berry extracts of the invention and compositions derived therefrom include, but are not limited to, atherosclerosis, arteriosclerosis, xanthomatosis, hyperlipoproteinemias, and familial and hypercholesterolemia.

Thrombotic diseases and conditions that may be treated using berry extracts of the invention and compositions derived therefrom include, but are not limited to, pulmonary disease (for example, involving reduced conductance, compliance, or constriction), excessive fluid accumulation or pulmonary edema, respiratory distress, asthma, pulmonary vascular permeability, pulmonary vasoconstriction, pulmonary hypertension, pulmonary embolism, cardiac ischemia, myocardial infarction, cardiopulmonary bypass associated dysfunction, vasoconstriction, organ dysfunction, platelet dysfunction, cardiac disease, chronic obstructive arterial disease caused by arteriosclerosis, vasoconstriction, renal artery stenosis, myocardial infarction, stroke, deep vein thrombosis, peripheral arterial occlusion, and other blood system thromboses.

Antiatherogenic diseases and conditions that can be treated using berry extracts of the invention and compositions derived therefrom include, but are not limited to, atherosclerosis, arteriosclerosis, myocardial infarction, ischemia (i.e., myocardial ischemia, brain ischemia, and renal ischemia) and strokes.

Inflammatory diseases and conditions that can be treated using berry extracts of the invention and compositions derived therefrom include, but are not limited to, essential hypertension, hypertension of congestive heart failure, renal dysfunction caused by reduced myocardia output, endotoxemia, chronic liver disease or hypertension, pulmonary inflammation in asthma, lung injury (bronchitis, pneumonia, or acute); rheumatic diseases (for example, rheumatoid arthritis or systemic lupus erythematosus), inflammatory bowel disease (for example, ulcerative colitis), irritable bowel disease (such as villous adenoma), gastrointestinal disorders caused by excess acids, pepsin or bile salts, skin diseases or trauma (such as burns or acid or caustic injury), rheumatoid diseases.

Immunoregulatory diseases and diseases that can be treated using berry extracts of the invention and compositions derived therefrom include, but are not limited to, autoimmune diseases, for example, AIDS, chronic fatigue syndrome, graft rejections, and other viral diseases that impair the immune system.

It is understood that the extracts of the invention (and fractions thereof) are capable of inhibiting any of the diseases or conditions described herein through the modulation, for example, via its antioxidant activity, of one or more mechanisms. Such mechanisms include, modulation of a chemical carcinogen prior to its metabolism or contact with a cell; modulation of the metabolism of a carcinogen, modulation of a carcinogen metabolite (e.g., by scavenging or binding to the metabolite before it can cause oxidative damage of a lipid, protein, or genetic material); and/or modulation of a cellular pathway (e.g., signal transduction or gene transcription).

### Synergy with Other Components Derived From Berry Extracts and/or Exogenous Compounds

In another embodiment, berry extracts of the invention, or one or a combination of components derived therefrom, are administered to a subject with an additional (exogenous) compound, e.g., an anti-cancer agent such as a chemotherapeutic compound and/or in combination with, for example, radiotherapy for the treatment of cancer. Administration of berries or their fractions along with chemotherapeutic drugs can permit more long-term, low-dose treatment of cancer patients with chemotherapy. In addition, patients treated with radiotherapy can obtain some protection against the harmful effects of radiation on normal tissues since these effects can be attributed largely to oxidative damage.

Accordingly, the berry extracts of the invention and compositions derived therefrom (particularly those having antioxidant activity) can be used alone or in combination with chemotherapeutic agents (including radiotherapy) as potent anti-cancer agents.

### Formulations and Methods of Administration

The berry extracts of the invention and compositions derived therefrom can be administered to a subject in any suitable form. For example, the extracts and compositions of the invention are sufficiently stable such that they can be readily prepared in a form suitable for adding to various foodstuffs including, for example, juice, fruit drinks, carbonated beverages, milk, nutritional drinks (e.g., Ensure™, Metracal™), ice cream, breakfast cereals, biscuits, cakes, muffins, cookies, toppings, bread, bagels, fiber bars, soups, crackers, baby formulae (e.g., Similac™), teas, salad dressings, cooking oils, and meat extenders. The berry extracts of the invention may also be delivered in the form of jellies, jams, or preserves.

In addition, berry extracts of the invention and compositions derived therefrom can be formulated as a pharmaceutical composition (e.g., a medicinal drug) for the treatment of specific disorders.

In another embodiment, berry extracts of the invention and compositions derived therefrom can be formulated as a dietary supplement.

Suitable additives, carriers and methods for preparing such formulations are well known in the art.

For example, pharmaceutical compositions may take the form of tablets, capsules, emulsions, suspensions and powders for oral administration, sterile solutions or emulsions for parenteral administration, sterile solutions for intravenous administration and gels, lotions and cremes for topical application. The pharmaceutical compositions may be administered to humans and animals in a safe and pharmaceutically effective amount to elicit any of the desired results indicated for the compounds and mixtures described herein. In addition, the extracts of the invention may be used in cosmetics.

The pharmaceutical compositions of this invention typically comprise a pharmaceutically effective amount of a berry extract or fraction thereof containing, for example, a berry extract with antioxidant activity, and, if suitable, a pharmaceutically acceptable carrier. Such carriers may be solid or liquid, such as, for example, cornstarch, lactose, sucrose, olive oil, or sesame oil. If a solid carrier is used, the dosage forms may be tablets, capsules or lozenges. Liquid dosage forms include soft gelatin capsules, syrup or liquid suspension.

Therapeutic and prophylactic methods of this invention comprise the step of treating patients or animals in a pharmaceutically acceptable manner with the compositions and mixtures described herein.

The pharmaceutical compositions of this invention may be employed in a conventional manner for the treatment and prevention of any of the aforementioned diseases and conditions. Such methods of treatment and prophylaxis are well-recognized in the art and may be chosen by those of ordinary skill in the art from the available methods and techniques. Generally, dosage ranges may be from about 1 to about 1000 mg/day. However, lower or higher dosages may be employed. The specific dosage and treatment regimens selected will depend upon factors such as the patient's or animal's health, and the severity and course of the patient's (or animal's) condition and the judgment of the treating physician. In certain embodiments, a diet is formulated to include the freeze dried berry powders of the invention in a concentration from about 1% to about 25% by weight. In a preferred embodiment, the concentration is about 5% by weight. In another preferred embodiment, the concentration is about 10%. In yet another preferred embodiment, the concentration is about 15%. In still another embodiment, the concentration is about 20%.

The berry extracts of the invention and compositions derived therefrom also can be used in combination with conventional therapeutics used in the treatment or prophylaxis of any of the aforementioned diseases. Such combination therapies advantageously utilize lower dosages of those conventional therapeutics, thus avoiding possible toxicity incurred when those agents are used alone. For example, other nutrients or medications, for example, cholesterol lowering drugs, chemotherapeutic agents, and/or radiotherapy.

In foodstuffs, the berry extracts of the invention and compositions derived therefrom can be used with any suitable carrier or edible additive. For example, the berry extracts of the invention may be used in foodstuffs, such as baked goods (for example, breads, muffins, and pastries), and cereals. The berry extracts of the invention and compositions derived therefrom also can be emulsified and used in a variety of water-based foodstuffs, such as drinks, for example, juice drinks, sports drinks, and drink mixes. Advantageously, the above-mentioned foodstuffs may be included in low fat, low cholesterol, or otherwise restricted dietary regimens.

Pharmaceutical compositions, dietary supplements, and foodstuffs of the present invention can be administered to humans and animals such as, for example, livestock and poultry.

This invention is further illustrated by the following examples which should not be construed as limiting.

### Exemplification

Throughout the examples, the following materials and methods were used unless otherwise stated.

Materials and Methods

In general, the practice of the present invention employs, unless otherwise indicated, conventional techniques of chemistry, e.g., food chemistry. Other techniques for carrying out the invention, for example, for preparing fruit extracts (and fractions thereof) and performing animal or cell-based assays for determining the anti-cancer properties of an extract (or fractions thereof), can be found, for example, in: Carlton et al., Carcinogenesis, 22:441-446 (2001); Stoner et al., Tox. Sciences Supp. 95-100 (1999); Carlton et al., Cancer Letter, 159:113-117 (2000); Xue et al., Carcinogenesis, 22:351-356 (2001); Harris et al., Proc. Amer. Assoc. Can. Res., pg 177 (Abstract) (2001); Xue et al., Tox. Sciences Supp., 54:267 (Abstract) (2000); Kresty et al., Proc. Amer. Assoc. Can. Res., 40:59 (Abstract) (1999); Kresty et al., Proc. Amer. Assoc. Can. Res., 39:18 (Abstract) (1998); Stoner et al., Proc. Amer. Assoc. Can. Res., 38:367 (Abstract) (1997); Kresty, et al., Can. Res. 61:6112-6119 (2001); Huang et al. Proc. Natl. Acad. Sci. USA. 95:156-161, (1998), and Casto et al., Anticancer Research, (in press) (2002).

Preparation of Extracts and Fractions Thereof

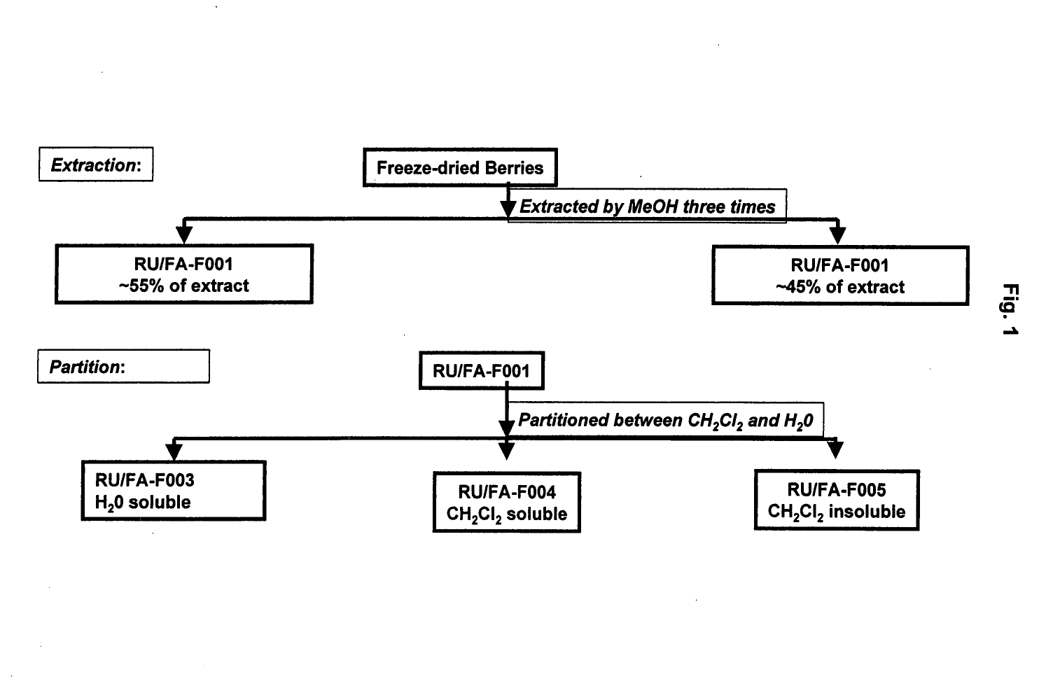

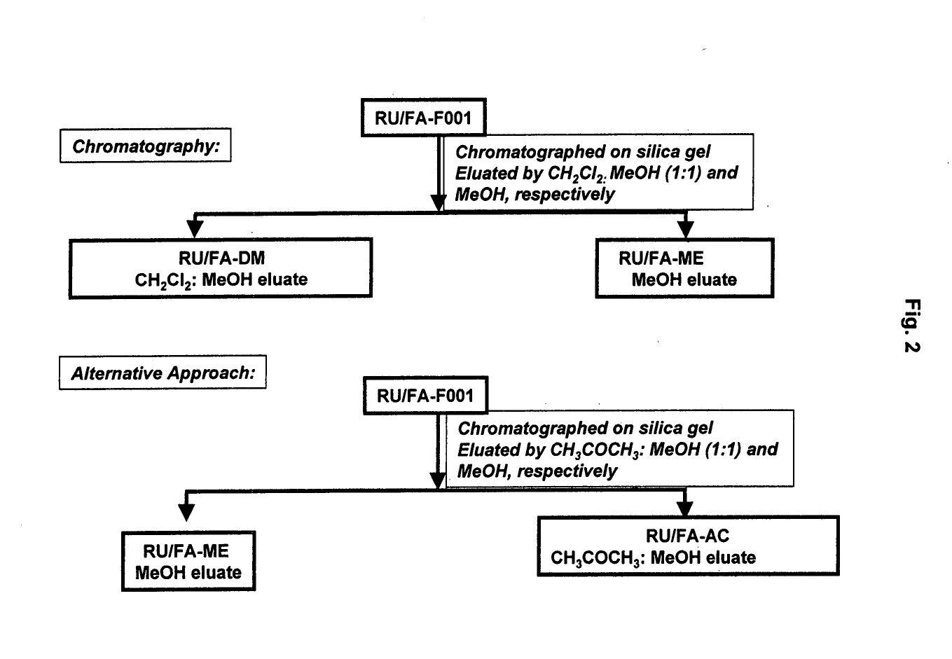

Preparation of extracts by freeze-drying was carried out as described in the examples using standard techniques. Techniques for solvent extraction of desirable fractions of the extracts (referred to as extract fractions) were carried out as described in the examples (e.g., Example 3) and as diagrammed in the figures of the application (FIG. 1-2). The alcohol fraction isolated using methanol (ME) is isolated using ethanol (Et) in some studies. Ethanol is a preferred extraction vehicle for extracts intended for human administration.

Analysis of Extract/Fraction Antioxidant Activity

Antioxidant activity was typically measured as a function of the oxygen radical absorbance capacity (ORAC) of the sample which was determined using standard techniques. Briefly, in the ORAC assay mixture, B-PE was used as a target of free radical change, MPH as a peroxyl radical generator, and Trolox as a control standard, and fluorescence (at, e.g., the following wavelengths of 540 nm (excitation) and 565 nm (emission)), was measured after addition of MPH to the extract or fraction (e.g., as described in Wang et al., J. Agric. Food Chem., 44:701-705 (1996); and Cao et al., J. Nutr.128:2383-90 (1998)).

For measuring the antioxidant activity of an extract of the invention (or fraction thereof) in altering the levels of particular radicals, electron spin resonance (ESR) spin trapping measurements can be made using standard techniques (e.g., as described in Leonard et al., J. of Environ. Path., Tox., and Onc. 19:49-60 (2000)).

Analysis of Extract/Fraction Components

Typically, compounds from the berry extracts of the invention are analyzed using art-recognized techniques such as solvent extraction and HPLC analysis. Components of the extracts, for example, carotenoids, phenolic compounds, phytosterols can be extracted and analyzed using, for example, thin layer chromatography and high-performance liquid chromatography. For example, the material can be fractionated on thin-layer chromatography (TLC) plates where the individual bands that are subsequently resolved can be scraped and extracted with a chloroform/methanol solvent. These resultant samples can then be analyzed using, e.g., gas and high-performance liquid chromatography (HPLC).

Other methods known in the art may also be employed, in place of or in combination with, the methods described above for isolating berry extract components, particularly to “scale up” the quantity of the isolated components. For example, chromatographic techniques may be used for isolating components of the berry extracts of the invention, in sufficient and pure quantities, such that the component may be administered alone or as part of a composition or product described herein (e.g., foodstuffs, dietary supplements, pharmaceuticals, etc.).

In particular, gas liquid chromatography, gas solid chromatography, high pressure or high performance liquid chromatography (HPLC) (e.g., normal, reverse, or chiral), ion exchange chromatography, or size exclusion chromatography can be employed as described, for example, in Advances in Chromatography, Brown, Eds., Marcel Dekker, Pub. (1998); Basic Gas Chromatography, Harold et al., John Wiley & Sons, Pub. (1997); Column Handbook for Size Exclusion Chromatography, Wu, Ed., Academic Press, Pub. (1999); Fundamentals of Preparative and Nonlinear Chromatography, Guichon et al., Eds., Academic Press, Pub. (1994); Handbook of Process Chromatography: A Guide to Optimization, Scale-Up and Validation, Hagel et al., Eds., Academic Press, Pub. (1997); HPLC Methods for Pharmaceutical Analysis, Lunn et al., John Wiley & Sons, Pub. (1997); and Practical High-Performance Liquid Chromatography, Meyer, Wiley-Liss, Pub. (1999), each of which is incorporated by reference herein.

Animal Assays

Animal assays were carried out using art-recognized techniques as described in the examples, and for example, as described in: Carlton et al., Carcinogenesis, 22:441-446 (2001) and Carlton et al., Cancer Letters, 159:113-117 (2000).

In animal studies featuring hamsters, Male Syrian Golden hamsters (Mesocricetus auratus), 3-4 weeks of age, were obtained from the Charles River Laboratories (Wilmington, Mass.). Three animals each were placed in plastic bottom cages with hardwood chip bedding and allowed to acclimate for one week. Food (AIN-76A, a modified semi-synthetic, high starch diet, Dyets Inc., Bethlehem, Pa.) and water were given ad libitum with the AIN-76A powdered diet provided in rat feeding jars. Animals were weighed weekly during berry extract and carcinogen treatment. All of the experimental conditions were in accordance with NIH Guidelines and with protocols approved by The Ohio State University Animal Care and Use Committee.

Cancer Inducing Agents for Animal Models

The agent 7,12-dimethylbenz(a)anthracene (DMBA) was obtained from Sigma-Aldrich (Milwaukee, Wis.) and dissolved at an 0.2% concentration in dimethylsulfoxide (DMSO) obtained from Fisher Scientific, Pittsburgh, Pa. 4-(methylnitrosamino)-1-(3-pyridyl)-1-butanone (NNK) was obtained from Toronto Research Chemicals, Ontario, Canada and benzo(a)pyrene (BaP) from Sigma Chemical Co., St. Louis, Mo. The BaP/NNK mixture was prepared at a 1% concentration in DMSO. The agent NMBA, obtained from Ash Stevens Corp., Detroit, Mich., and azoxymethane, obtained from Sigma Chemical Co., were purified by HPLC to greater than 98% purity.

Diet Preparation for Animal Models

For most studies, black raspberries (Jewel variety) were supplied by the Dale Stokes Berry Farm (Wilmington, Ohio) and shipped frozen to Van Drunen Farms (Momence Ill.) for freeze-drying. The composition of the berry powder (extract) was determined by Covance Laboratories (Madison, Wis.) and is presented in Table 1 along with the composition of two other lyophilized black raspberry (LBR) extracts used for inhibition of rat esophageal tumors. The LBR powder was mixed into a modified AIN-76A diet at 5% and 10% concentrations with the concentration of cornstarch adjusted to maintain an isocaloric diet among all experimental groups. Berry-containing and control diets were prepared every two weeks, 140-170 grams measured into pint rat feeding jars, and stored at 4° C. Two jars were placed into each cage, feeding jars were rotated with each jar being replaced every 5-8 days with fresh feed, and the before and after weights of the jars recorded.

Diets comprising 5% and 10% lyophilized black raspberries (LBR) were prepared as described above and determined to comprise the following components as indicated in Table 1.

TABLE 1
Composition of Cultivars of Black Raspberry Extracts
used in Chemoprevention Studies

| | Identification of berry lot | | |
|---|---|---|---|
| Components^{a} | LBR98^{b} | LBR95^{c} | LBR97^{d} |

Minerals

| | | | |
|---|---|---|---|
| Calcium | 170.00 | 245.00 | 215.00 |
| Copper | 0.74 | 0.52 | 0.55 |
| Iron | 4.95 | 13.20 | 10.10 |
| Magnesium | 147.00 | 169.00 | 153.00 |
| Manganese | 5.85 | 3.60 | 4.68 |
| Phosphorus | 168.00 | 222.00 | 170.00 |
| Potassium | 1060.00 | 1200.00 | 1300.00 |
| Sodium | <10.00 | <10.00 | <10.00 |
| Zinc | 2.12 | 2.69 | 2.12 |
| Selenium | <5.00 | <5.00 | <5.00 |

Vitamins

| | | | |
|---|---|---|---|
| Folic Acid | 0.51 | 0.07 | 0.06 |
| Vitamin C | <1.00 | <1.0 | 4.14 |

Sterols

| | | | |
|---|---|---|---|
| _-sitosterol | 72.40 | 89.10 | 80.10 |
| Campesterol | 4.60 | 4.30 | 3.40 |
| Cholesterol | <1.00 | <3.00 | <1.00 |
| Stigmasterol | <3.00 | <1.00 | <3.00 |

Phenolics

| | | | |
|---|---|---|---|
| Ellagic Acid | 200.00 | 185.00 | 166.30 |
| Ferulic Acid | 21.00 | 32.40 | 17.60 |
| p-Coumaric | 6.72 | 7.94 | 9.23 |
| Acid | | | |

Carotenoids

| | | | |
|---|---|---|---|
| _-carotene | <0.02 | <0.02 | <0.02 |
| _-carotene | 0.12 | <0.02 | <0.02 |
| Lutein | <0.02 | <0.02 | <0.02 |
| Zeaxanthin | <0.02 | <0.02 | <0.02 |

^{a}Concentration of components is expressed as mg/100 g of LBR: selenium is expressed as _g/100 g.
^{b}Lot used for inhibition of oral tumors in HCP.
^{c}Lot used for inhibition of esophageal tumors in rats (complete carcinogenesis bioassay, Kresty et al., Cancer Res. 61: 6112-6119 (2001)
^{d}Lot used for inhibition of esophageal tumors (post -initiation assay; ref. #46)

Induction of Tumors and Chemoprevention Protocol for Animal Models

Three groups of 15 hamsters, 3-4 weeks of age, were placed on diets containing either 0, 5%, or 10% LBR. Two weeks later (5-6 weeks of age), the three groups (Table 5) were treated with carcinogen according to a modification of the initial methods as described (Morris et al., J. Dental Res. 40:3-15 (1961)). Hamsters were lightly anesthetized with Isoflurane and the opening of the pouch made accessible by inserting a small metal pegboard hook at the side of the mouth and gently pulling the hook laterally away from the hamster to expose the interior surface of the pouch. The three groups of animals were treated by painting both surfaces of each pouch 3 times weekly for 8 weeks with an 0.2% solution of DMBA dissolved in DMSO using a No.4 camel hair brush (Dachi et al., Cancer Res. 27:1183-1185 (1967)). Before, during, and after carcinogen treatment, Group 1 animals received a diet with 5% LBR, Group 2 received a diet containing 10% LBR, and Group 3 received the AIN-76A diet alone. Tumors of sufficient mass in the control group (3-10 mm in greatest length) suitable for final analyses appeared in 70-77 days (10 to 11 weeks) after beginning DMBA treatment. Twelve to thirteen weeks from the beginning of berry treatment and following CO₂ euthanasia, tumors were harvested, processed for evaluation, and final histologic examination performed after fixing and staining. Hematoxylin and eosin stained hamster cheek pouches were evaluated and histologically characterized by a board-certified oral pathologist in the College of Dentistry at The Ohio State University.

³²P-Postlabeling Assays for DMBA Adduct Analysis from Animal Model Tissues

Assays for determining DMBA induced adduct formation were typically carried out as follows. Two groups of six hamsters each were treated with a 5% concentration of black raspberry extract in a modified AIN-76A diet for two weeks. One day after cessation of berry treatment, both cheek pouches of each group were painted with an 0.2% solution of DMBA in DMSO. Six control animals without berry treatment were painted with 0.2% DMBA+DMSO in the right cheek pouch or DMSO alone in the left pouch. Twenty-four and 48 hrs after DMBA or DMSO treatment, the animals were sacrificed by CO₂ euthanasia and the pouches quick frozen in liquid nitrogen.

DNA was isolated from the left and right cheek pouch tissue of each animal using a direct salt-precipitation method (Miller et al., Nucleic Acid Res. 16:1215 (1988); Schut et al., Cancer Lett. 67:117124 (1992)). ³²P-postlabeling assays for DMBA-DNA adducts were run under intensification conditions (Randerath et al., Carcinogenesis 6: 1117-1126 (1985)). The assay conditions were identical to those used before (50), except for the D3 solvent that was used for the initial separation of adducts (3.5 M lithium formate, 7.0 M urea, pH 3.5) and the D5 solvent (1.0 M magnesium chloride). DNA adduct levels were expressed as relative adduct labeling (RAL) values, after correction of the <RAL>values obtained under intensification conditions.

Induction of Dysplasia in Animal Models: Tissue Collection and Analysis

Carcinogen-induced tissue dysplasia studies were typically carried out as follows. Hamster cheek pouches were painted with 0.2% DMBA in DMSO or DMSO alone 3×/wk for 3 weeks or 3×/wk for 10 weeks with 1% BaP/NNK or DMSO alone. At 3 weeks (DMBA or DMSO) or at 4, 7 and 10 weeks (BaP/NNK or DMSO), cheek pouches from animals that were treated with carcinogen or solvent were harvested and cut longitudinally. One section of each cheek pouch was immediately frozen in liquid nitrogen and stored at −80° C. A second portion of the pouch was fixed in 10% neutral buffered formalin for no more than 8 hrs and paraffin embedded on edge in separate paraffin blocks.

Serial 4 μm sections were cut from formalin-fixed pouches and mounted on Superfrost Plus slides (Fisher Scientific, Pittsburgh, Pa.). A hematoxylin and eosin slide of each HCP was prepared and random tissue sections from each animal were scanned at 10OX magnification by an oral pathologist. Each view in field was categorized into one of four histologic categories: normal epithelium, epithelial hyperplasia, low-grade dysplasia, or high-grade dysplasia. The classification scheme utilized was modified from criteria developed by Pozharisski et al. (Tumors of the Esophagus, IARC Scientific Publications, Lyon (1973), pp 87-100) with consideration toward the gross and microscopic descriptions of hyperplasia and dysplasia given in Robbins: Pathologic Basis of Disease. 5th edition.

Statistical Analysis in Animal Models

Differences between berry fed groups and the control group in the number of tumors were analyzed using Kendall's tau statistics (equivalent to the Mann-Whitney test corrected for ties). In addition, the Fisher exact test was used to examine the dichotomy of having a high versus low number of tumors per animal. The DNA adduct data was evaluated for statistical significance using an ANOVA model accounting for harvest time and LBR. Since a proportionate, rather than absolute, change in response was expected, the response variable in the ANOVA model was on the log scale.

Cell-Based Assays

Cell-based assays were carried out using art-recognized techniques as described in the examples, and for example, as described in Xue et al., Carcinogenesis, 22:351-356 (2001).

In studies featuring cell lines with reporter genes, typically mouse epidermal cells (i.e., JB-6 clone 41) were stably transfected with either an AP-1-luciferase reporter gene construct (P⁺1-1 cells), a NFκB-luciferase reporter gene construct (Cl 41 NFκB mass1 cells), or a p53-luciferase reporter gene construct (Cl 41 PG13 mass1 cells) (see, e.g., Huang et al., PNAS 94:11957-11962 (1997); Cancer Res. 57:2873-2878 (1997); and Int J Oncol 13:711-715 (1998)). These resultant cell lines (e.g., Cl 41, P⁺1-1, Cl 41 NFκB mass1 and Cl 41 PG13 mass1) were cultured in Eagle's Minimal Essential Medium (Calbiochem, San Diego, Calif.) supplemented with 5% fetal bovine serum (FBS), 2 mM L-glutamine, and 25 μg of gentamicin/ml (Life Technologies, Inc., Rockville, Md. Cells were cultured at 37° C. in a humidified atmosphere of 5% CO₂ in air. The cultures were dissociated with trypsin and transferred to new 75 cm² culture flasks (Fisher, Pittsburgh, Pa.) from one to three times per week. The substrate for the luciferase assay was obtained from Promega (Madison, Wis.); BPDE was obtained from Sigma (St. Louis, Mo.); and the phospho-specific antibodies against various phosphorylated sites of ERKs, p38 kinase, JNKs, and IκBα were obtained from New England Biolaboratories (Beverly, Mass.). The radiolabel (±)-r-7, t-8-dihydroxy-t-9,10-epoxy-7,8,9,10-tetrahydro[1,3-³H]benzo[a]pyrene ([³H]-BPDE, specific activity, 2210 mCi/mmol) was obtained from ChemSyn Science Laboratories (NCI Chemical Carcinogen Repository, Kansas City, Mo.).

AP-1 ActivitV Assay

The AP-1 activity assay was typically performed using confluent monolayers of P⁺1-1 cells cultured under standard conditions and subsequently incubated with different fractions of black raspberry extract dissolved in DMSO for 30 min at concentrations ranging from 1-100 μg/ml. Cells were then exposed to BPDE at a final concentration of 2 μM. The cells were extracted with lysis buffer (Promega, Madison, Wis.) at various periods of time (6-48 h) after BPDE exposure, and the luciferase activity was determined by the Luciferase assay using a luminometer (Wallac 1420 Victor 2 multilable counter system) after the addition of lysis buffer. The results are expressed as AP-1 activity relative to control medium containing DMSO (0.1% v/v) only (Relative AP-1 activity).

NFκB and p53-Dependent Transcription Activity Assays

The same procedure as described above for measuring the effects of berry fractions on BPDE-induced AP-1 activity in P⁺1-1 cells was used for determining the effects of the same berry fractions on BPDE-induced NFκB activity in NFκB mass1 cells, and p53-dependent transcription activity in PG-13 mass1 cells. The results were expressed as either NFκB activity or p53-dependent transcription activity relative to control medium containing DMSO.

Kinase Phosphorylation Assay

Immunoblots were performed with either phospho-specific antibodies or non-phosphorylated antibodies against various kinases, including ERKs, JNKs and p38 kinase, and also against IκBα. The protein band specifically bound to the primary antibody was detected using an anti-rabbit IgG-AP-linked and an ECF immunoblotting system (Amersham Biosciences, Piscataway, N.J.).

### EXAMPLE I

### Method of Preparing Berry Extracts

The following studies were performed to determine methods for isolating a berry extract having desirable properties or producing an extract being enriched for particular components.

In brief, freeze-dried (lyophilized) black raspberries (Rubus occidentalis) and strawberries (Fragaria ananassa) were made as follows. Several hundred pounds of fresh, ripe black raspberries and strawberries were picked, washed, and stored frozen at −20° C. Berry puree, free of cap stems and seeds, was prepared by passing the whole berries through a pulper-finisher fitted with a screen having 0.020-inch perforations. The waste fraction was returned to the pulper three times to assure complete juicing of the harder white shoulders of the berries. The seed was pulverized and added to the puree. The puree containing pulverized seed was poured to a depth of approximately 1 inch into freeze-dryer trays lined with polyethylene film, and then frozen in a blast freezer. The frozen plates of puree were removed and stored at −20° C. for subsequent freeze drying.

Freeze-drying was accomplished by means of a Virtis model 50-SRC-5 Sublimator. The shelf temperature was 40° C. and the vacuum was 380 millitorr. One defrost cycle was required for each batch containing about 70 pounds of puree. Approximately three days are required to dry each batch of puree. When dry, the thickest portion of each plate of dried material was visually checked for remaining ice. If ice was found, freeze-drying was continued. When the product was found to be dry, it was packaged in doubled polyethylene bags, placed in carton boxes, and stored at −20° C.

The berry extracts were then used as the source material for further analysis and fractionation described below.

### EXAMPLE 2

### Analysis of the Components of Berry Extracts

In this example, the berry extracts, isolated using the methods of the invention described above, were subjected to a detailed analysis of its beneficial components.

In particular, samples of freeze-dried strawberries and black raspberries prepared as described above where analyzed for their overall antioxidant activity as well as the presence of selected vitamins, carotenoids, phenolic compounds, phytosterols and minerals.

First, the overall antioxidant activity for each extract was determined using techniques described herein and results are shown in Table 2.

TABLE 2
Oxygen Radical Absorbance Capacity (ORAC)

| Extract | ORAC Value (per mg) |
|---|---|
| Strawberry | 15.36* |
| Black Raspberry | 16.09 |

*= estimate based on Wang et al. (Agr. Found. 44: 701-705 (1996)).

The content of selected vitamins, carotenoids, phenolic compounds, phytosterols, and minerals, was then determined for several extract samples of each fruit. Strawberry extracts (prepared from fresh strawberries or strawberries that were frozen at −20° C. for either 24 hours or several months after picking) were analyzed and results are shown in Table 3. All strawberry extract components in Table 3 were well preserved for a period of at least one year after the berries were freeze-dried and maintained at either −20° C. or at refrigerator temperature (4° C.). An exception is vitamin C. The vitamin C in fresh strawberries is well preserved in freeze-dried material if the berries are freeze-dried within 24 hours after harvesting. In contrast, when the berries are stored frozen at −20° C. for several months after harvesting, the vitamin C content is markedly reduced. Thus, the vitamin C in strawberries degrades rapidly when the berries are stored for several months at −20° C. before freeze-drying.

TABLE 3
Components of Strawberry Extracts

| | | | |
|---|---|---|---|
| | Fresh | Freeze-Dried | Freeze-Dried |
| | Strawberries | Strawberries | Strawberries |
| Component | (mg/kg)^{a} | (mg/100 g)^{a} | (mg/100 g)^{b} |

Vitamins

| | | | |
|---|---|---|---|
| Vitamin A | 425.00 | 267.00 | —^{c} |
| Vitamin E | 5.86 | 495 | —^{c} |
| Vitamin C | 348.50 | 371.00 | 141.00 |
| Folic Acid | 0.60 | 0.59 | 0.37 |

Carotenoids

| | | | |
|---|---|---|---|
| α-carotene | <0.02 | <0.02 | 0.03 |
| β-carotene | 0.25 | 0.16 | <0.02 |
| Zeaxanthin | <0.02 | <0.02 | <0.02 |
| Lutein | 0.17 | 0.11 | <0.02 |

Phenolic compounds

| | | | |
|---|---|---|---|
| Ellagic acid | —^{c} | 67.00 | 140.00 |
| Ferulic acid | <2.50 | <2.5 | <2.5 |

Phytosterols

| | | | |
|---|---|---|---|
| β-sitosterol | 39.10 | 40.70 | 27.20 |
| Campesterol | <3.00 | <3.00 | <3.00 |
| Stigmasterol | <3.00 | <3.00 | <3.00 |

Minerals

| | | | |
|---|---|---|---|
| Calcium | 92.65 | 72.40 | 160.00 |
| Magnesium | 109.65 | 91.90 | 124.00 |
| Potassium | 1445.00 | 1110.00 | 1640.00 |
| Zinc | 0.93 | 0.63 | 0.99 |
| Selenium | <0.01 | <0.01 | 11.00 |

Fiber

| | | | |
|---|---|---|---|
| Fiber | ˜5% of total wt. | ˜45% of total wt. | ˜45% of total wt. |

^{a}November 2000 harvest. Fresh strawberries were frozen at −20° C. immediately after purchase from a store. Some were kept frozen and the remaining berries were freeze-dried. Both the fresh berries and freeze-dried berries were analyzed for various components
^{b}October 1995 harvest. Stored frozen several months before freeze drying. Then stored in a refrigerator for one year before analysis.
^{c}not analyzed
^{d}approximate value

In Table 4, the content of selected vitamins, carotenoids, phenolic compounds, phytosterols, and minerals, in black raspberry extracts is shown. Some of these data were presented in Table 1. In both samples, the black raspberries were stored frozen at −20° C. for at least six months before they were freeze-dried. As indicated, the vitamin C content of the black raspberries is low suggesting that it degraded during storage at −20° C. The contents of the other berry components is well preserved for more than one year when stored at refrigerator temperature (4° C.).

TABLE 4
Components of Black Raspberry Extracts

| | | |
|---|---|---|
| | Black Raspberries | Black Raspberries |
| Substance | (mg/100 g)^{a} | (mg/100 g)^{b} |

Vitamins

| | | |
|---|---|---|
| Vitamin A | —^{c} | —^{c} |
| Vitamin E | —^{c} | 10.80 |
| Vitamin C | <1.00 | <0.10 |
| Folic Acid | 0.07 | 0.013 |

Carotenoids

| | | |
|---|---|---|
| α-carotene | <0.02 | <0.02 |
| β-carotene | <0.02 | 0.012 |
| Zeaxanthin | <0.02 | <0.04 |
| Lutein | <0.02 | 0.03 |

Phenolic Compounds

| | | |
|---|---|---|
| Ellagic acid | 175.00 | 200.00 |
| Ferulic acid | 32.40 | 21.00 |
| Anthocyanins | —^{c} | 1770.00 |

Phytosterols

| | | |
|---|---|---|
| β-sitosterol | 89.10 | 72.40 |
| Campesterol | 4.30 | 4.60 |
| Stigmasterol | <3.00 | <3.00 |

Minerals

| | | |
|---|---|---|
| Calcium | 245.00 | 167.00 |
| Magnesium | 169.00 | 147.00 |
| Potassium | 1200.00 | 1060.00 |
| Zinc | 2.70 | 2.12 |
| Selenium | <5.00 | <0.01 |

Fiber

| | | |
|---|---|---|
| Fiber | ˜45% of total freeze | ˜45% of total freeze |
| | dried wt. | dried wt. |

^{a}December 1995 harvest. Stored frozen several months before freeze-drying. Then stored in refrigerator for one year before analysis.
^{b}July 1998 harvest. Stored frozen several months before freeze-drying. Then stored in refrigerator for one year before analysis.
^{c}not analyzed

For each fruit extract tested, beneficial compounds such as vitamins (e.g., Vitamin E, Vitamin C, and folic acid); carotenoids (e.g., _-carotene, β-carotene, zeaxanthin, lutein); phenolic compounds (e.g., ellagic acid, ferulic acid, and anthocyanins); phytosterols (e.g., β-sitosterol, campesterol, and stigmasterol, and analogs thereof); and minerals (e.g., calcium, magnesium, potassium, zinc, and selenium) were detected. The raspberry extracts of the invention are particularly enriched for the presence of antioxidant activity.

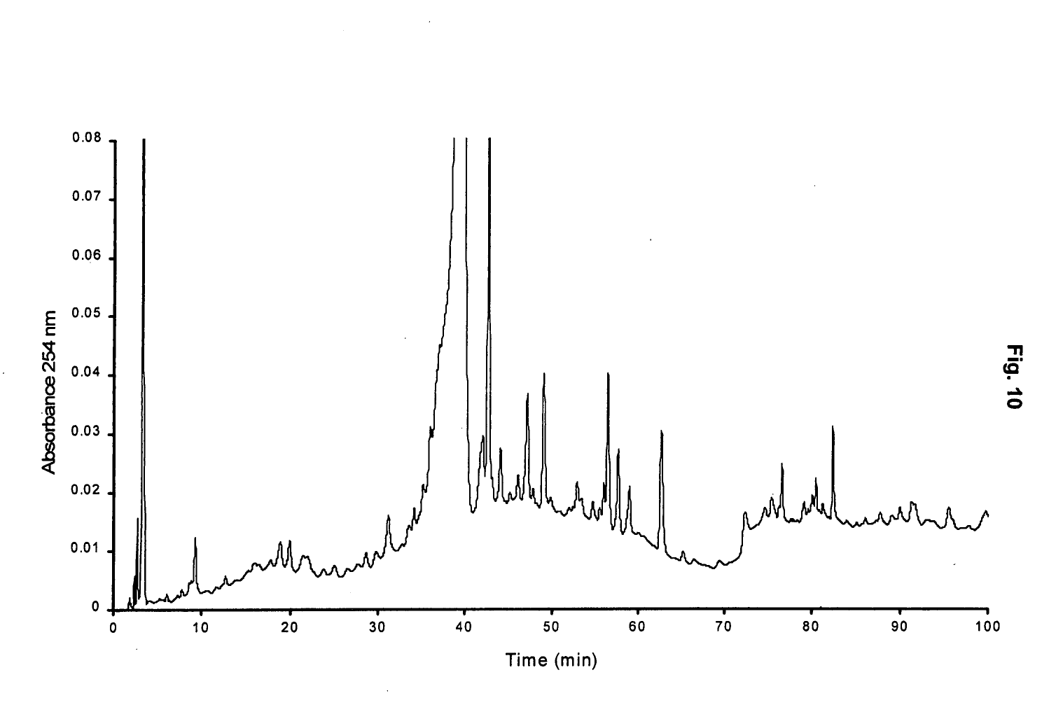

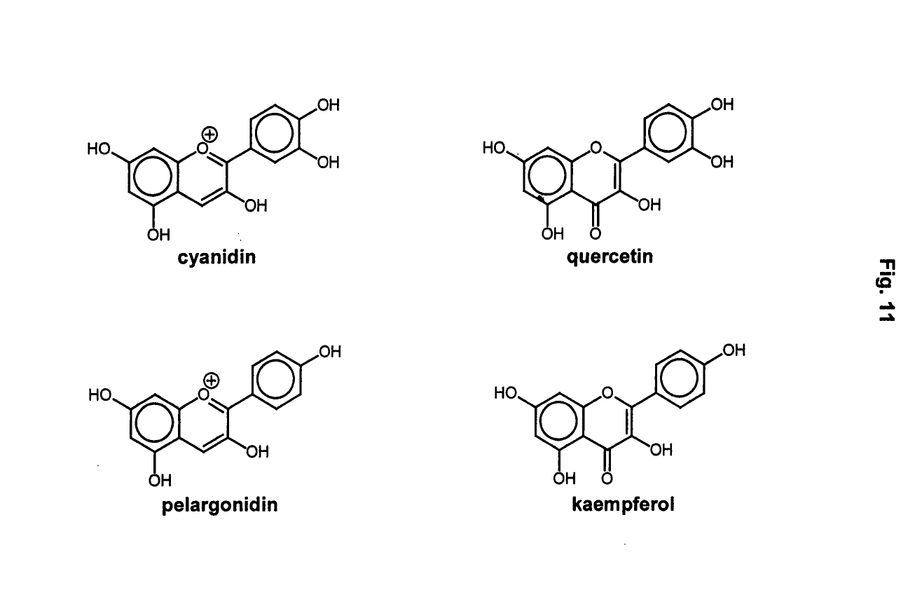

In addition, upon further fractionation of the black raspberry extracts in particular, several bioactive components were identified. Specifically, the methanol extract of freeze-dried black raspberries was further studied, as this fraction had the most activity in inhibition of cellular transformation and down regulation of AP-1 and NF_B activities, as discussed herein. Analysis of this fraction by HPLC with UV detection, using a C18 reverse-phase system, gave the chromatogram illustrated in FIG. 10. Using diode-array detection, UV spectra were obtained on all peaks. Liquid chromatography-electrospray ionization-negative ion-mass spectrometry (LC-ESI-MS) analysis of this fraction, with selected ion monitoring for the glycosides of 4 flavonoids known to be present in raspberries: cyanidin, quercetin, pelargonidin, and kaempferol, was carried out. The structures of these compounds are illustrated in FIG. 11.

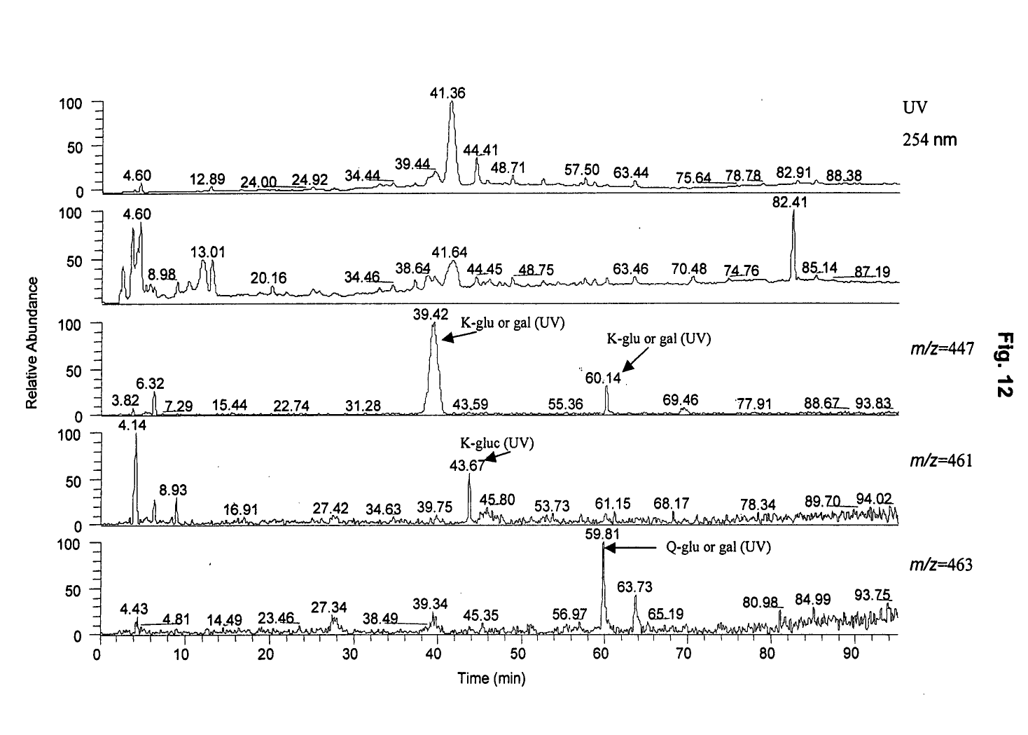

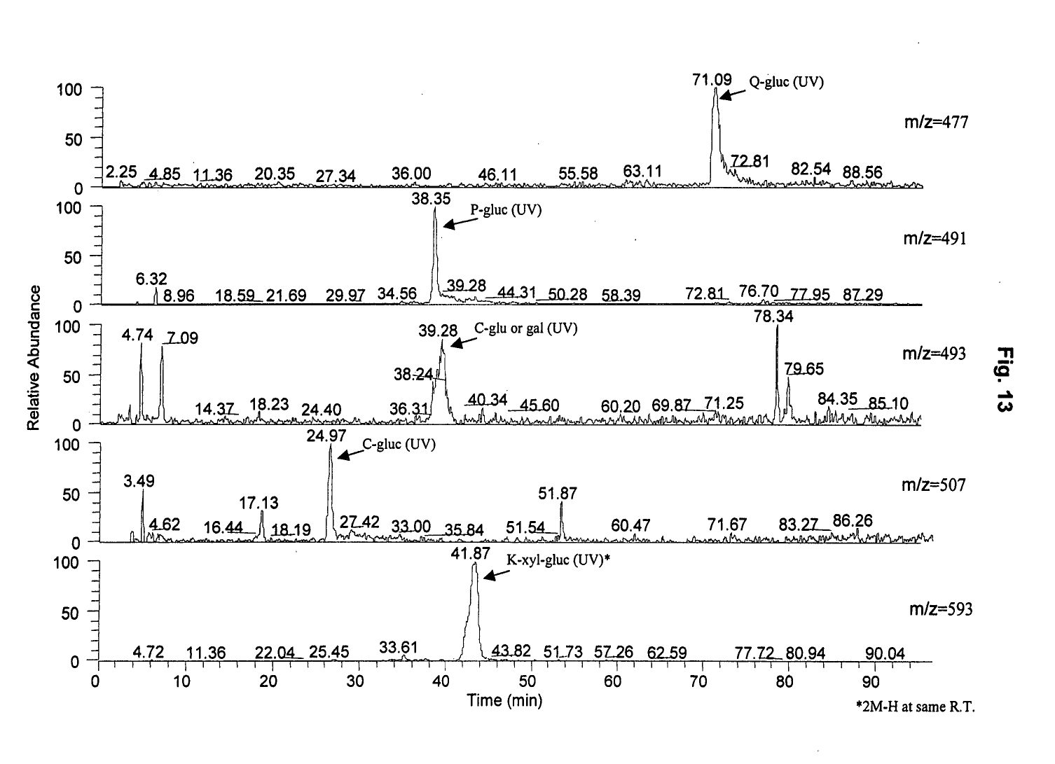

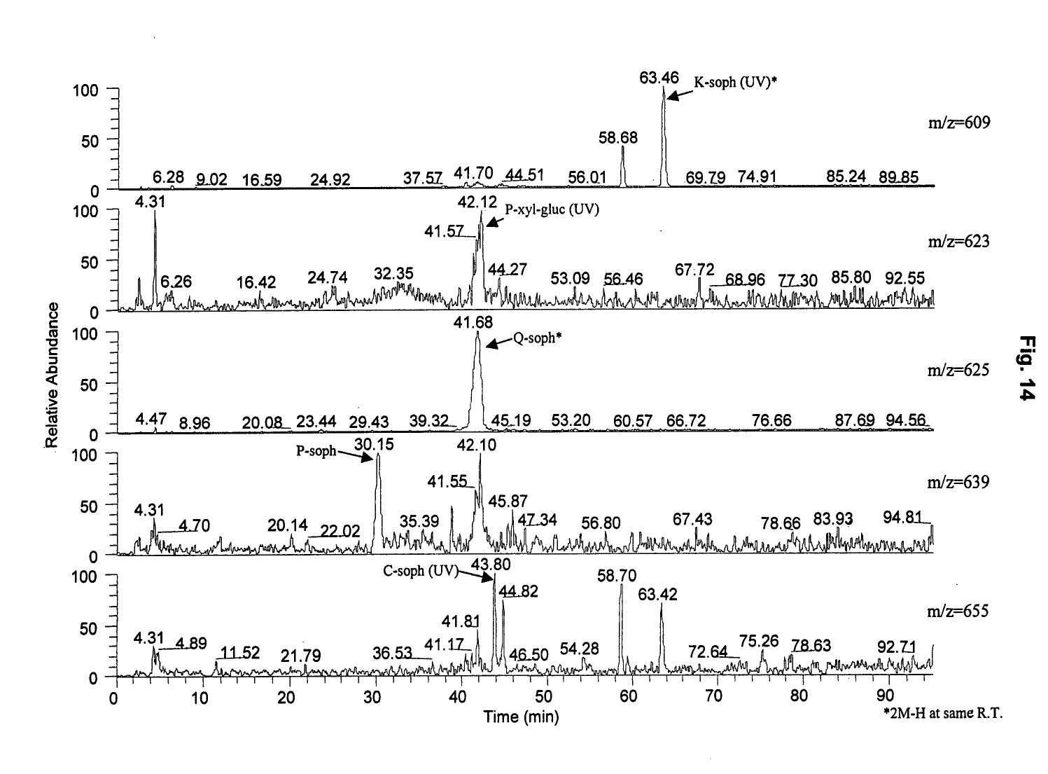

Analyses of standards demonstrated that M-1 peaks and 2M-1 peaks would be obtained under these conditions. In addition, the following known sugar conjugates of cyanidin, quercetin, pelargonidin, and kaempferol: glucoside, galactoside, glucuronide, sophoroside, and xylosylglucuronide were selected for ion monitoring analysis as shown in FIGS. 12-14. The top panel of FIG. 12 shows the UV trace, and the second panel shows the chromatogram obtained when monitoring total ion current. The third panel shows the chromatogram obtained by monitoring m/z 447, M-1 of kaempferol glucoside and galactoside. The peaks marked K-glu or gal (UV) also had UV spectra and MS consistent with kaempferol glucoside. Similarly, the fourth panel shows the results of selected ion monitoring for m/z 461, which is M-1 of kaempferol glucuronide. The peak marked K-gluc (UV) had UV and MS consistent with kaempferol-glucuronide. The fifth panel shows selected ion monitoring for m/z 463, M-1 of quercetin glucoside or galactoside. The peak marked Q glu or gal (UV) had UV and MS consistent with queretin glucoside or galactoside. FIGS. 13 and 14 show similar data, where P refers to pelargonidin and C to cyanidin.

Collectively, these data demonstrate the presence of desirable flavonoids in the active fraction of freeze-dried black raspberries.

### EXAMPLE 3

### Method of Fractionating Berry Extracts

In this example, methods for performing solvent extractions and fractionations of the berry extracts of the invention are described.

The protocol for the preparation of fractions from extracts of freeze-dried black raspberries and strawberries is shown in FIGS. 1 and 2. Typically, 400 grams of freeze-dried berries were extracted in 4,000 ml of methanol (or ethanol) at room temperature overnight. This procedure was repeated at least three times. The extracts were then concentrated under vacuum at a temperature below 60° F. This yields a fraction designated either RU-001 (from black raspberries) or FA-001 (from strawberries) that typically represents approximately 55% of the starting freeze-dried material.

The RU-001 or FA-001 fractions were then further extracted using three procedures. In brief, the first procedure was performed as follows. Using a separatory funnel, the fractions were partitioned between two volumes of water and two volumes of dichloromethane three times at room temperature. This yielded a water soluble fraction, designated either RU-003 or FA-003, and a dichloromethane soluble fraction, designated either RU-004 or FA-004. In a typical experiment, extraction of 335 g of RU-001 or FA-001 by this procedure yielded 275 g of RU-003 or FA-003 and 10 g of RU-004 or FA-004.

In another approach, the RU-001 or FA-001 fractions were chromatographed on a silica gel column using dichloromethane:methanol (1:1) and methanol eluates, respectively. The fraction eluted with dichloromethane:methanol is termed the DM fraction (i.e., RU-DM or FA-DM), and that eluted with methanol is termed the ME fraction (i.e., RU-ME or FA-ME).

In yet another approach, the RU-001 or FA-001 fractions were chromatographed on a silica gel column using acetone:methanol (1:1) and methanol eluates, respectively. The fraction eluted with methanol is termed the ME fraction (i.e., RU-ME or FA-ME), and that eluted with acetone:methanol was termed the AC fraction (i.e., RU-AC or FA-AC).

The various fractions derived from either freeze-dried black raspberries or strawberries were determined to have desirable health promoting activities as described below.

### EXAMPLE 4

### Method of Demonstrating the Anti-Cancer Properties of a Strawberry Extract

The following studies were performed to examine the anti-cancer properties of the strawberry extract of the invention.

Briefly, strawberry extracts (freeze-dried strawberries) prepared as described above were evaluated for their ability to inhibit chemically-induced tumors in rodents. Using the rat model of squamous cell carcinoma of the esophagus, strawberry extracts added at 5% and 10% of the diet 2 weeks before, during, and after subcutaneous administration of the chemical carcinogen, N-nitrosomethylbenzylamine (NMBA), caused significant reductions in the development of both preneoplastic lesions (simple hyperplasia, low- and high-grade dysplasia) and the number of esophageal tumors per rat by 24 and 56%, respectively. In addition, strawberry extracts added at 5% and 10% of the diet were shown to influence the metabolism of NMBA to DNA damaging species as indicated by the observation that they reduced the formation of O⁶-methylguanine adducts in esophageal DNA by 59 and 64%, respectively. When added at 5% and 10% of the diet following subcutaneous treatment of rats with NMBA (i.e., post-carcinogen treatment), the strawberry extracts also significantly reduced tumor multiplicity by 38 and 31%, respectively. Thus, the strawberry extracts were capable of suppressing the conversion of premalignant esophageal cells to malignant cells.

In contrast to the data in the esophagus, freeze-dried strawberries added at 10% of the diet one week before, during and after the administration of the tobacco carcinogens, benzo(a)pyrene or 4-(methylnitrosamino)-1-(3-pyridyl)-1-butanone (NNK) failed to inhibit lung tumor development in strain A/J mice. The lung tumor assay in A/J mice, is a model for adenocarcinoma of the human lung. These results suggest that the inhibitory components in strawberries do not reach the lung in sufficient quantities to protect against chemically-induced lung cancer when the strawberries are fed in the diet.

### EXAMPLE 5

### Method of Demonstrating the Anti-Cancer Properties of a Black Raspberry Extract

The following studies were performed to examine the anti-cancer properties of the black raspberry extract of the invention.

Briefly, freeze-dried black raspberries prepared as described above were evaluated for their ability to inhibit chemically-induced tumors in rodents. Using the rat model of squamous cell carcinoma of the esophagus, freeze-dried black raspberries added at 5% and 10% of the diet 2 weeks before, during, and after subcutaneous administration of the carcinogen, N-nitrosomethylbenzylamine (NMBA) caused significant reductions in esophageal tumor multiplicity of 39 and 49%, respectively. These reductions in tumor multiplicity are very similar to what was obtained with freeze-dried strawberry extracts. In addition, black raspberry extracts added at 5% and 10% of the diet were shown to influence the metabolism of NMBA to DNA damaging species as indicated by the observation that they reduced the formation of O⁶-methylguanine adducts in esophageal DNA by 73 and 80%, respectively. When added at 5% and 10% of the diet following subcutaneous treatment of rats with NMBA, the black raspberries significantly reduced the formation of both premalignant lesions (i.e., low- and high-grade dysplasia) and the number of esophageal tumors per rat by 62 and 43%, respectively. In addition, the berries were shown to reduce the rate of proliferation of premalignant cells as evidenced by significant reductions in the percentage of cells stained positively for proliferating cell nuclear antigen (PCNA). Thus, one mechanism by which black raspberries inhibit tumor progression in the rat esophagus is by reducing the rate of growth of epithelial cells in the esophagus of NMBA-treated animals.

In another study, freeze-dried black raspberries were evaluated for their ability to inhibit the progression of chemically-induced cancer in the rat colon. Rats were given intraperitoneal injections of the colon carcinogen, azoxymethane, once per week for two weeks. One day after the final injection, rats were administered 2.5%, 5%, and 10% freeze-dried black raspberry extracts in the diet. After 33 weeks of dietary administration, 2.5, 5 and 10% black raspberry extracts, reduced total colon tumor numbers (adenoma and adenocarcinoma) by 42, 45, and 71%, respectively. In addition, in the same treatment groups, the number of adenocarcinomas decreased by 28, 35, and 80%, respectively. This is very significant because adenocarcinomas represent malignant tumors in the colon of rats and, also, of humans. All reductions in tumor number were statistically significant. This study also revealed that urinary 8-hydroxydeoxyguanosine (8-OHdG) levels were reduced by 73, 81 and 83%, respectively, in rats administered 2.5, 5 and 10% berry extracts in the diet. Therefore, black raspberry extracts also modulated an important marker of oxidative stress in azoxymethane-treated rats.

In another study, the anti-oral cancer properties of the black raspberry extracts of the invention, and fractions derived therefrom, were examined.

In particular, the hamster cheek pouch (HCP) animal model was used to evaluate the ability of black raspberries to inhibit oral cavity tumors. Male Syrian Golden hamsters, 3-4 weeks of age, were fed 5% and 10% lyophilized black raspberries (LBR) in the diet for two weeks prior to treatment with a cancer inducing agent (ie., 0.2% 7,12-dimethylbenz(a)anthracene in dimethylsulfoxide; hereafter DMBA) and for 10 weeks thereafter.

Diets comprising 5% and 10% black raspberry extracts were prepared as described above and determined to comprise the components indicated in Table 1 (page 24).

The cancer agent was applied to the oral cavities of the animals for eight weeks after which the animals were sacrificed 12-13 weeks from the beginning of DMBA treatment (Table 5) and the number and volume of tumors (mm³) was determined (Table 6). There was a significant difference (p=0.02) in the number of tumors observed between the 5% black raspberry extract and control groups (27 tumors/14 animals and 48 tumors/15 animals, respectively) and an intermediate number of tumors was observed in the 10% berry-treated animals (39 tumors/15 animals). These experiments show that dietary black raspberries will inhibit tumor formation in the oral cavity.

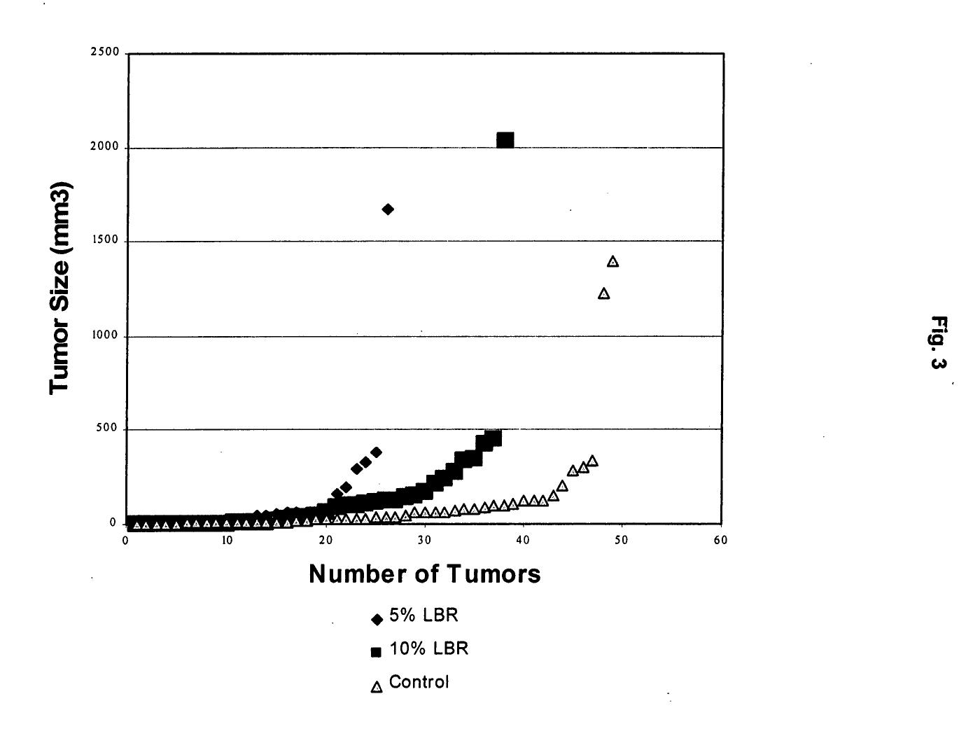

The inhibition of oral cancer, i.e., cheek pouch tumors both in size and numbers by lyophilized black raspberries is shown in FIG. 3. There was no difference in tumor size or incidence between the three groups at time of sacrifice. Except for one animal in the 5% berry group all of the hamsters had at least one tumor arise in one or both pouches. However, only 9 of the 29 animals (31%) ingesting berries had 3 or more tumors compared with 10 of the 15 (67%) hamsters in the control group (p=0.03). Moreover, there were significantly fewer tumors per animal in the 5% LBR group (Table 6) when compared to the control group (27 tumors in 14 animals treated with 5% LBR and 48 tumors in 15 animals in the control group, p=0.02). The 15 animals treated with 10% lyophilized black raspberries had an intermediate number of tumors (39 tumors in 15 animals). No statistically significant differences in body weight or food consumption were observed between the control animals and animals given test diets comprising 5% or 10% lyophilized black raspberries.

TABLE 5
Carcinogen initiation and chemoprevention in vivo protocol*

| Group | Wk. 1-2 | Wk. 2-8 | Wk 9-10 | Wk. 12-13 |
|---|---|---|---|---|
| 1 (N = 15) | 5% LBR | DMBA 3×/ | 5% LBR diet | Tumor harvest |
| | | wk + | | |
| | | 5% LBR diet | | |
| 2 (N = 15 | 10% LBR | DMBA | 10% LBR diet | Tumor harvest |
| | | 3×/week + | | |
| | | 10% LBR diet | | |
| 3 (N = 15 | Control | DMBA | Control diet | Tumor harvest |
| | diet | 3×/week + | | |
| | | control diet | | |

*DMBA in DMSO solvent was administered via cheek pouch painting for 8 weeks (3×/week) beginning 2 weeks after LBR diets were started. Hamsters were given 5% and 10% LBR diets during the 8 weeks of DMBA treatment and for two weeks following treatment. At 12-13 weeks, animals in the DMBA treated and control groups (Groups 1, 2 and 3) were sacrificed and cheek pouches analyzed for tumors, tumor size, and histopathologic changes.

TABLE 6
Inhibition of hamster cheek pouch tumors by dietary consumption of LBR

| Group | | | # Tumors/ | Tumor |
|---|---|---|---|---|
| (N = 15) | Treatment^{a} | % Incidence^{b} | # Animals^{c} | Multiplicity |
| 1 | DMBA + 5% | 93% (13/14) | 27/14 | 1.93^{d} |
| | LBR | | | |
| 2 | DMBA + 10% | 100% (15/15) | 39/15 | 2.60 |
| | LBR | | | |
| 3 | DMBA control | 100% (15/15) | 48/15 | 3.20 |

^{a}Hamsters were given LBR in the diet for 2 weeks, treated with DMBA and LBR for 8 weeks, and given LBR in the diet for 2 additional weeks.
^{b}Cumulative number of tumors in treated animals. One animal in Group 1 died of unknown cause.
^{c}Total tumors in both pouches. Nine of 29 animals in the berry groups had less than 3 tumors, whereas 10/15 animals in control group had more than 3 tumors (p = 0.03)
^{d}significantly different than DMBA control (p = 0.02)

### EXAMPLE 6

### Method of Demonstrating the Cholesterol Lowering Activity of a Berry Extract

The following studies were performed to examine the cholesterol lowering activity of the berry extracts of the invention.

Briefly, blood analyses of animals (i.e., laboratory rats) fed black raspberry extract added at 5% to their diets were conducted to determine the effects of the extract on blood lipid levels. Blood samples were collected at 33 weeks, placed in heparinized tubes and analyzed by Antech (Alsip, Ill.) using standard techniques. Importantly, black raspberry extract consumption at 5% of the diet significantly reduced blood cholesterol levels from 248.76±44.63 mg/dl in the diet control group to 223.57±44.81 mg/dl in the group administered the extract. The berry extract had no effect on other blood lipid values. This same method can be applied to determine the cholesterol lowering activity of the strawberry extracts of the invention.

Thus, berry extracts (e.g., black raspberry) of the invention have significant cholesterol lowering potential as demonstrated using a relevant animal model.

### EXAMPLE 7

### Method of Demonstrating the Berry Extracts are Non-Toxic

The following studies were performed to show that the berry extracts of the invention are non-toxic when administered to a mammal.

During the bioassays (described above) to evaluate the efficacy of freeze-dried strawberry extracts to inhibit chemically-induced tumors in the rat esophagus and mouse lung, and of black raspberry extracts to inhibit chemically-induced tumors of the rat esophagus and colon, weight and food consumption data were collected on a weekly basis. In the mouse lung study, the data were collected for 16 weeks; in the rat esophagus studies, 25 to 35 weeks; and, in the rat colon study, 33 weeks. Data from control animals of the foregoing studies were collected (i.e., from animals administered either a control diet (without berry extracts) or diets containing 10% of either strawberry extracts or black raspberry extracts. The results showed that neither strawberry extract nor black raspberry extract produced toxic effects in the animals as defined by reduced food consumption or weight loss. Animals fed berry extracts consumed about the same amount of food and weighed approximately the same as animals administered a control diet.

In addition, various organs were collected at necropsy from rats that had been fed 5% or 10% strawberry or black raspberry diets in the bioassays and evaluated for any gross pathology. The organs collected (e.g., esophagus, stomach, small intestine, colon, liver, kidney, bladder, spleen, heart and lungs) were also fixed in 10% neutral buffered formalin for subsequent histopathological analysis. Histopathological examination of these tissues revealed no abnormal changes that could be associated with consumption of either strawberry or black raspberry extracts.

### EXAMPLE 8

### Cell-Based Method Demonstrating the Anti-Cancer Properties of Berry Extract Fractions

The following studies were performed to examine the anti-cancer properties of the berry extracts of the invention and fractions derived therefrom.

Briefly, black raspberry extract fractions (RU-F001, RU-F003, RU-F004, RU-F005, RU-DM, RU-ME) and strawberry extract fractions (FA-F001, FA-F003, FA-F004, FA-F005, FA-DM, FA-ME) isolated as described above were analyzed for anti-transformation activity in the Syrian hamster embryo (SHE) cell transformation model using benzo(a)pyrene (B[a]P) as the chemical carcinogen. None of the extract fractions by themselves produced an increase in morphological transformation. For assessment of chemopreventive activity, SHE cells were treated with each extract fraction at doses ranging from 2-100 microgram per milliliter and B[a]P (10 microgram per milliliter) for seven days. The RU-ME and FA-ME extract fractions isolated as described above produced a dose-dependent decrease in transformation as compared to B[a]P treatment only.

The raspberry extract fraction (RU-ME) and strawberry extract fraction (FA-ME) were further examined using a 24 hour co-treatment with B[a]P or a 6 day treatment following a 24 hour treatment with B[a]P. Both extract fractions significantly reduced B[a]P-induced transformation when co-treated with B[a]P for 24 hours. These results indicate that the methanol fractions from black raspberry extracts and strawberry extracts inhibit cell transformation through interference of the uptake, activation and/or detoxification of B[a]P and/or intervention of DNA binding and DNA repair.

### EXAMPLE 9

### Method of Demonstrating the Anti-Oxidant Properties of a Berry Extract

The following studies were performed to examine the antioxidant activity of the berry extracts of the invention.

In particular, the oxygen radical absorbance capacity (ORAC) assay was performed to test for the presence of antioxidant activity. Both freeze-dried black raspberry and strawberry extracts were tested for antioxidant activity using the ORAC assay and as indicated in Table 3. The ORAC values for both berry types was elevated.

In another approach for determining the antioxidant activity of the berry extracts of the invention, electron spin resonance technology was used. Each fraction was evaluated for its ability to quench singlet oxygen and hydroxide ion (electron spin resonance (ESR)). The fractions exhibit varying abilities to quench these free radicals, and overall, are highly active when compared to control compounds with high levels of antioxidant activity.

These results indicate that both the berry extracts and fractions derived therefrom have desirable antioxidant activity.

### EXAMPLE 10

### Methods of Demonstrating that Berry Extracts Modulate Signal Transduction

The following studies were performed to demonstrate the molecular mechanisms involved in the inhibition of carcinogenesis by a berry extract of the invention.

Accordingly, a black raspberry extract was investigated for its ability to modulate transactivation of AP-1 and NFκB induced by benzo(a)pyrene diol-epoxide (BPDE), the resultant carcinogen of B(a)P, in mouse epidermal cells (i.e., JB-6 clone 41 cells).

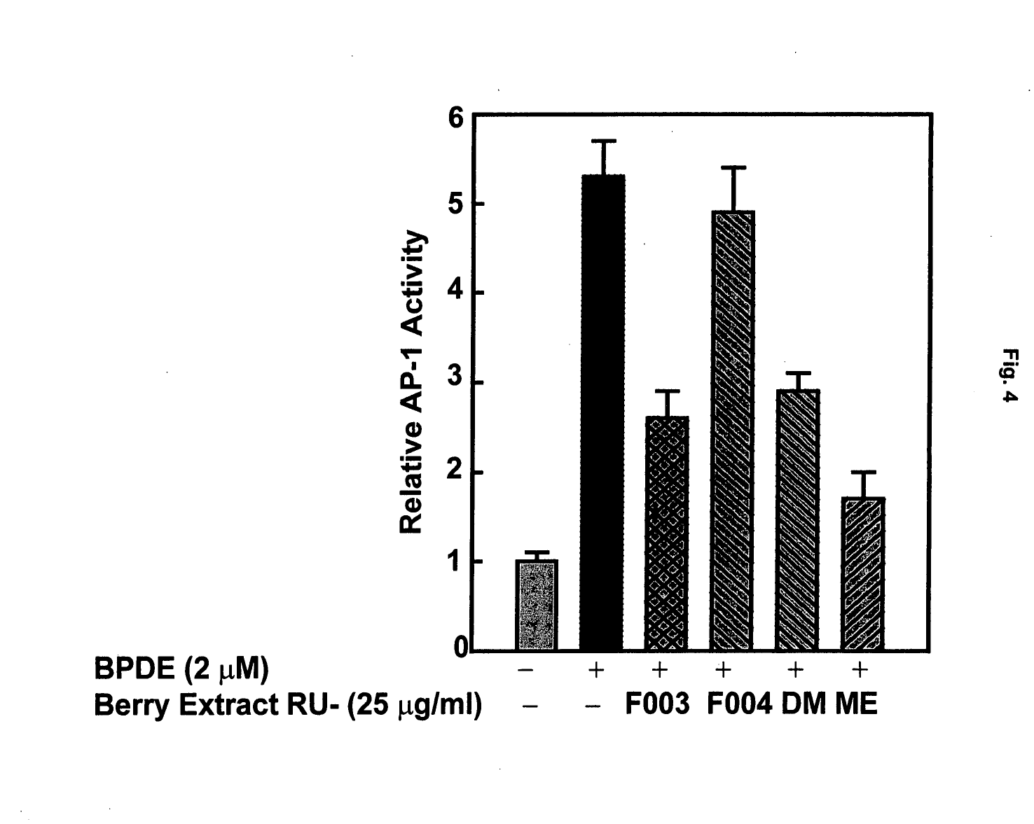

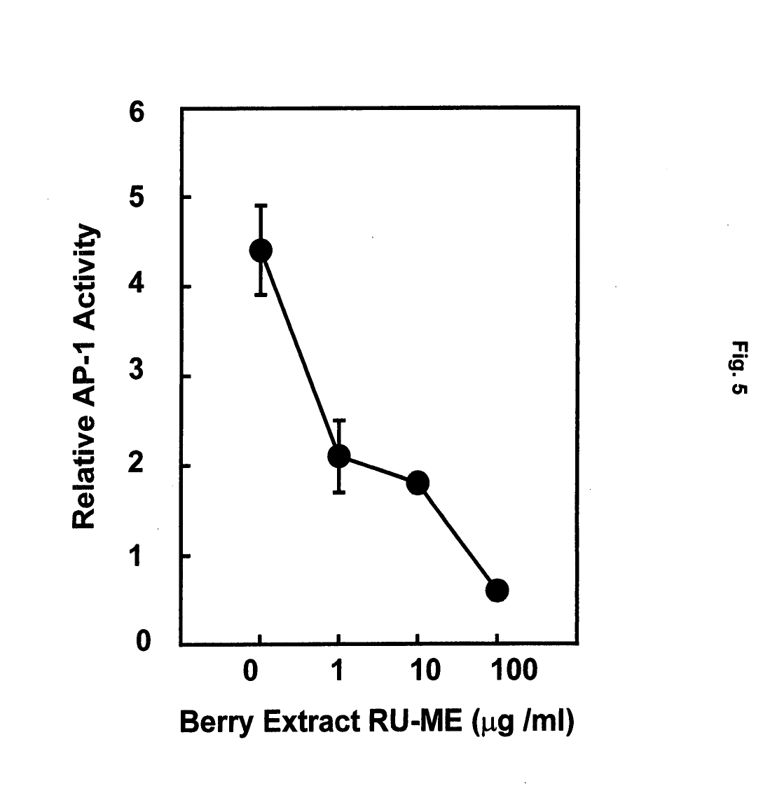

In particular, the potential effects of the black raspberry fractions (i.e., RU-F003, RU-F004, RU-DM, and RU-ME, see, e.g., FIGS. 1-2) on BPDE-induced AP-1 activation on mouse P⁺1-1 cells were examined. Specifically, P+1-1 cells were pretreated with each of four fractions (RU-F003, RU-F004, RU-DM and RU-ME) at 25 μg/ml for 30 min, and then exposed to 2 _M BPDE to induce AP-1. Pretreatment of P⁺1-1 cells with either the RU-F003, RU-DM or RU-ME fractions resulted in a significant inhibition (P<0.05) of BPDE-induced AP-1 activity, while the RU-F004 extract had no effect (FIG. 4). The RU-ME fraction was the most potent inhibitor of AP-1 activity among the extracts tested (FIG. 4), which is consistent with its potency as an inhibitor of B(a)P-induced cell transformation. The RU-ME fraction was inhibitory when added to the medium at only 1 μg/ml (FIG. 5).

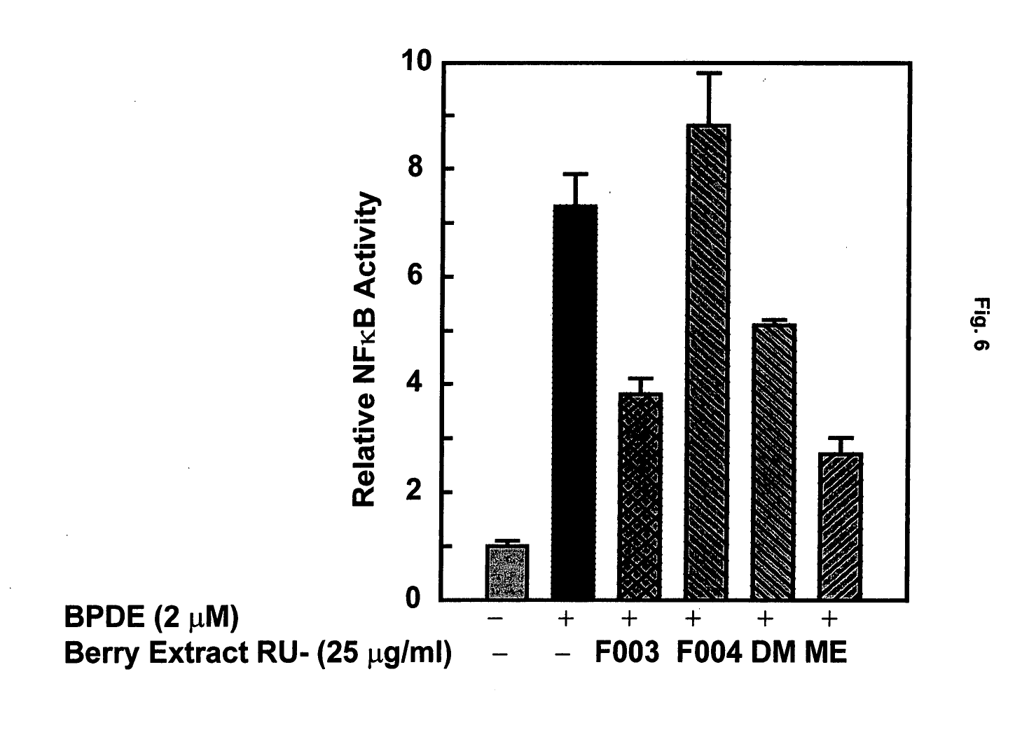

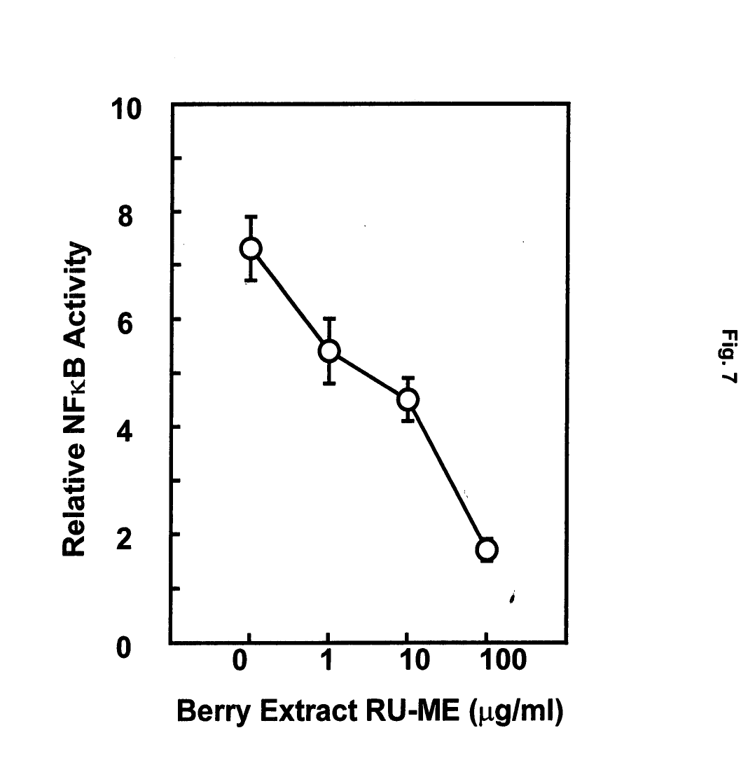

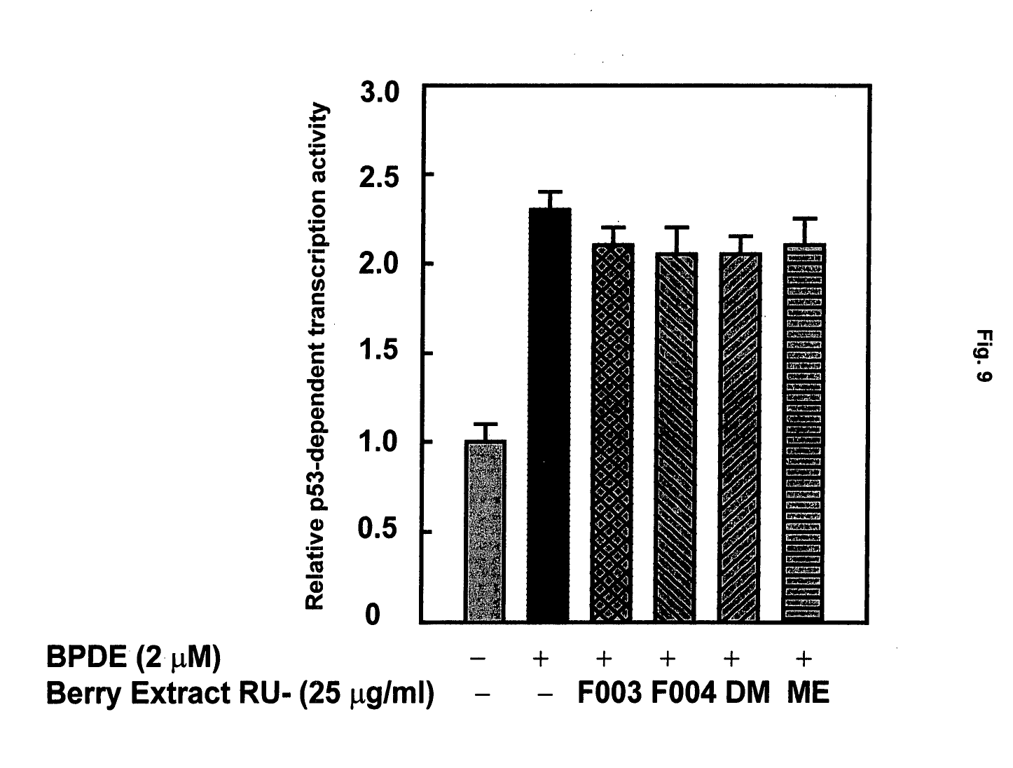

In another study, the effect of berry fractions on the induction of NFκB by BPDE in mass1 cells, was examined. Specifically, pre-incubation of the cells with either the RU-F003, RU-DM or the RU-ME fraction led to a significant inhibition (P<0.05) of BPDE-induced NFκB activity in the cells (FIG. 6). In contrast, fraction RU-F004 did not inhibit NFκB activity (FIG. 6). The RU-ME fraction was the most potent inhibitor of NFκB activity among the fractions tested (FIG. 6). The inhibitory effect of RU-ME on BPDE-induced NFκB activity was observed to be in dose- and time-dependent manner (FIG. 7). As seen in the studies above, BPDE-induced p53-dependent activation was not affected by any of the fractions tested on mass1 cells (FIG. 9).

To determine the conditions under which the berry fractions inhibit BPDE-induced activation of AP-1 and NFκB in Cl 41 cells, the most active fraction, RU-ME, was added to cultured Cl41 cells at different times before or after exposure of the cells to 2 μM BPDE. The inhibitory effect of the RU-ME fraction on both AP-1 and NFκB occurred only when RU-ME was added either before or along with the BPDE. RU-ME was not effective when added to the cells 3 hours after treatment with BPDE. These data indicate that pre-treatment or simultaneous co-incubation of RU-ME with BPDE is required for inhibition of BPDE-induced activation of AP-1 and NFκB.

Black raspberries contain multiple compounds with known chemopreventive activity. Among these, ellagic acid can react with BPDE to form covalently linked cis and trans adducts in which the reactive epoxide ring of the pyrene is open, rendering the BPDE harmless. In order to determine whether inhibition of BPDE-induced activation of AP-1 and NFκB by the RU-ME fraction might be due to a similar reaction of compounds in RU-ME with BPDE, the effect of RU-ME on BPDE-induced DNA adduct formation was tested. If compounds in RU-ME react with BPDE, then one might expect lowered levels of BPDE binding to Cl 41 cell DNA. To determine the effect of RU-ME on BPDE-DNA adduct formation, cultured Cl 41 cells were treated with [³H]-BPDE or [³H]-BPDE and RU-ME mixture. The ³H count in a known quantity of purified genomic DNA was determined. The number of BPDE-induced DNA adducts in a 10 kb genomic DNA fragment was then calculated. The results demonstrated that pre-incubation of the RU-ME fraction with BPDE did not reduce BPDE-DNA adduct formation in Cl 41 cells. Accordingly, the mechanism of action of the extracts is not by the binding of extract components to BPDE, which would inhibit BPDE binding to cellular DNA.

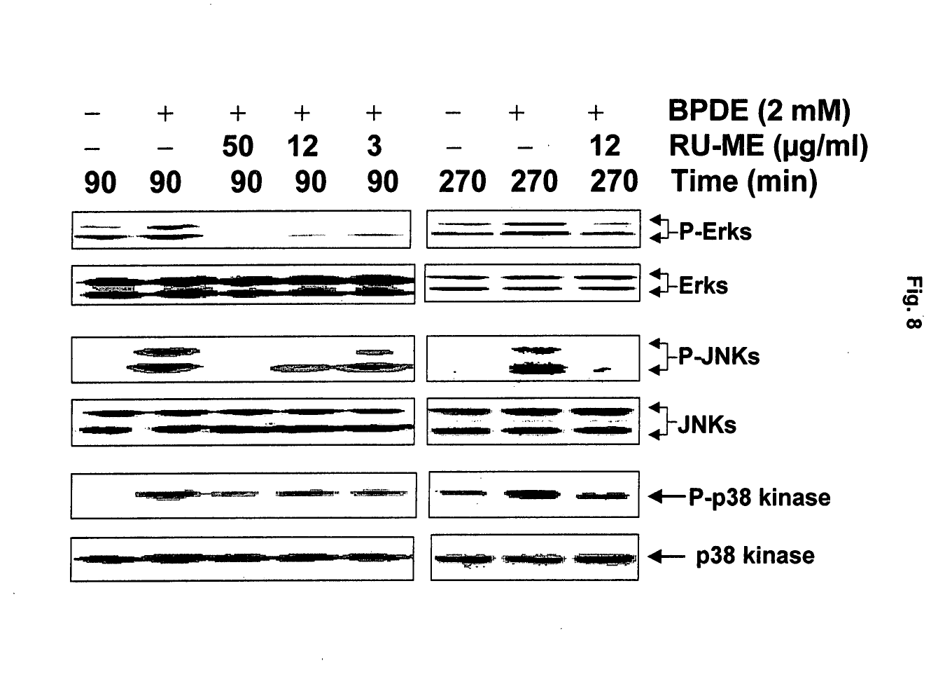

To test the effects of the RU-ME fraction on BPDE-induced activation of the ERKs, JNKs, and P38 kinases in Cl 41 cells, the effects of RU-ME on phosphorylation of the MAP kinase family were tested. The results showed that pretreatment of cells with RU-ME led to a significant inhibition of phosphorylation of ERKs, JNKs and p38 kinase (FIG. 8), indicating that all three MAP kinase family members are involved in the inhibitory effect of RU-ME on AP-1 activation.

To determine whether inhibition of BPDE-induced NFκB by RU-ME is caused by inhibition of IκBα phosphorylation and degradation, IκBα phosphorylation in cells exposed to BPDE and RU-ME using phospho-specific antibody was determined. Results obtained indicate that pretreatment of cells with RU-ME inhibited BPDE-induced increase in phosphorylation of IκBα at 90 min, and degradation of IκBα protein at 270 min, after BPDE treatment.

Thus, the RU-ME fraction was determined to be the most potent inhibitor of BPDE-induced AP-1 and NFκB activities among the fractions tested, which is consistent with its potency as an inhibitor of B(a)P-induced cell transformation. In addition, the inhibitory effects of RU-ME on BPDE-induced activation of AP-1 and NFκB can be mediated via inhibition of MAP kinase activity and IκBα phosphorylation, respectively.

Accordingly, in view of the important roles of AP-1 and NFκB in tumor promotion, these results indicate that RU-ME is a major fraction for chemopreventive activity in black raspberry extracts, and that the anti-tumor progression activity of black raspberries can be mediated by impairing signal transduction pathways leading to activation of AP-1 and NFκB.

### EXAMPLE 11

### In vivo Method Demonstrating Berry Extract Fractions Inhibit the Formation of DNA Adducts

The following studies were performed to examine the ability of the berry extracts of the invention to inhibit the formation of DNA adducts in vivo.

In this example, the hamster cheek pouch (HCP) animal model as described above was used to evaluate the ability of black raspberries to inhibit the formation of DNA adducts in the check pouches of animals treated with the cancer inducing agent, DMBA. Under intensification conditions and using the ³²P-postlabeling technique, a total of four DNA adducts could be detected in the cheek pouches of animals treated with DMBA. After running the assay under standard (ATP-saturating) conditions, intensification factors for adducts 1, 3, and 4 were found to be 37.7, 8.1, and 10.5, respectively. A minor adduct (#2) was not detectable under standard assay conditions as it amounted to only 1.2-4.3% of the total intensified adducts (<RAL> values), except for four separate samples where it constituted 7.1-9.8% of the total. Of the total corrected adducts (RAL values), adducts 1, 3, and 4 constituted 38.8-59.0%, 21.3-35.9%, and 17.8-29.0%, respectively, of the adduct burden. For quantitative comparisons, total RAL's (sum of adducts 1, 3, and 4) and sum of specific adducts were used. The 5% berry diet inhibited DMBA adducts by 29% and 55% (mean total adduct levels) at 24 and 48 hr (Table 7 below) with a statistical significance of p=0.07. Similar differences between berry and DMBA control groups for the formation of other adducts was observed.

TABLE 7
Inhibition of DMBA Adducts by Lyophilized Black Raspberries

| | | DNA adducts (RAL × 10⁷)^{c} | | | |
|---|---|---|---|---|---|
| Treatment^{a} | Harvest^{b} | Adduct 1 | Adduct 3 | Adduct 4 | Total SEM^{d} |
| 5% LBR + DMBA | 24 hr | 17.95 | 12.24 | 9.52 | 39.7 +/− 10.7 |
| Control + DMBA | | 26.50 | 15.88 | 13.09 | 55.5 +/− 16.2 |
| Control + DMSO | | 0.9441 | 0.4079 | 0.3467 | 1.70 +/− 0.34 |
| 5% LBR + DMBA | 48 hr | 10.84 | 6.87 | 5.52 | 23.2 +/− 11.8 |
| Control + DMBA | | 24.02 | 14.11 | 13.10 | 51.2 +/− 3.8 |
| Control + DMSO | | 0.5226 | 0.2129 | 0.2102 | 0.95 +/− 0.11 |

^{a}Hamsters were given 5% LBR or AIN-76A control diet for 48 hr prior to DMBA challenge: DMBA was given as a single dose by painting the HCP with 0.2% DMBA in DMSO.
^{b}Twenty-four and 48 hr after DMBA treatment, cheek pouches were harvested from euthanized animals and immediately frozen in LN₂.
^{c}Relative Adduct Labeling under intensification conditions (ref. 51). A minor DMBA adduct (#2) is not shown in the table, as it represented only 1.2-4.3% of the total intensified adducts.
^{d}Total adduct burden. Sum of RAL of adducts 1, 3, and 4″ ± standard error of mean.
e Overall difference between 5% berry treated and DMBA control animals was only marginally significant.

This study indicates one mechanism by which the berry extracts of the invention reduce cancer, i.e., tumor burden, is that the extracts (LBR) inhibit the formation of pro-mutagenic adducts formed by DMBA. In short term bioassays, feeding of both 5% and 10% berries prior to a single carcinogen treatment with 0.25 mg/Kg NMBA resulted in 73% and 80% reductions in O⁶-methylguanine adducts in esophageal tumorgenesis (Kresty et al., Cancer Res. 61: 6112- 6119, 2001.). In the HCP, when hamsters were given 5% LBR for two weeks prior to DMBA challenge, three major adducts (adducts 1,3,4) were found to be inhibited by 29% when analyzed 24 hr after DMBA treatment. When analyzed 48 hr after DMBA treatment, the inhibition of DMBA-DNA adducts by 5% berries was greater than 50%. Therefore, the observed decrease in HCP tumors can be explained, in part, by the inhibition of DNA adduct formation.

In summary, the chemoprevention studies presented herein show that incorporation of black raspberries in the diet will inhibit tumor formation in the oral mucosa of mammals.

### EXAMPLE 12

### In vivo Method for Determining the Anticancer Properties of Berry Extracts on Tobacco Related Oral Cancers

The following studies are performed to examine the ability of the berry extracts of the invention to inhibit the formation of cancer caused by tobacco use.

In order to develop an oral cancer model that would mimic the conditions found in human oral mucosa after being exposed to exogenous tobacco carcinogens (e.g., polycyclic aromatic hydrocarbons (PAHs) and nitrosoamines), the cheek pouch of a model animal (i.e., hamsters, hamster cheek pouch (HCP); 2-3 animals per group) was painted with a reduced total dose of DMBA (0.2% DMBA in DMSO, 3×/wk for three weeks) or with a 1% BaP/NNK mixture (3×/wk for 10 wk). Twenty-four hours after the final DMBA treatment or at 4, 7, and 10 wk of BaP/NNK treatment, hamsters were sacrificed and the HCPs were divided longitudinally into two sections, one for quick freezing and the second for histological examination. Control tissues, that were treated only with DMSO solvent, had a normal histologic appearance with a normal orthokeratin pattern and no evidence of a hyperproliferative or inflammatory response upon histological examination. Histopathologic examination of sections taken 24 hrs after the last DMBA treatment showed morphologic changes ranging from a mild inflammatory response to areas of focal dysplasia, as evidenced by abnormal cell maturation, increased mitotic figures, and cellular pleomorphism.

Control hamster cheek pouch epithelium treated for 10 weeks with the DMSO vehicle, showed a uniform histology characterized by a 3-4 epithelial cell thickness, lack of defined epithelial rete ridges, hyperorthokeratosis, and un-inflamed connective tissue upon histological examination. In contrast, within 7 weeks after three times/week of 1% NNK/BaP topical application, the surface epithelium showed a slight basilar hyperplasia, increased thickness of the spinous layer (acanthoid), and a mild chronic inflammatory cell infiltrate in the superficial connective tissue upon histological examination. Ten weeks after NNK/BaP application, the experimental animals showed histologic evidence of epithelial dysplasia similar to the dysplastic epithelial progression in human oral mucosa, i.e., maturational perturbations began in the basilar third of the hamster epithelium. These tissues, upon histological examination, also evidenced tear-dropped shaped epithelial rete ridges in conjunction with basilar hyperplasia, consistent with moderate epithelial dysplasia.

Accordingly, the above animal model is suitable for determining the cancer inhibiting properties of the extracts described herein. In particular, for evaluating the ability of black raspberries to inhibit oral cavity tumors caused by long term tobacco use. Male Syrian Golden hamsters, 3-4 weeks of age, can be fed 5% and 10% lyophilized black raspberries (LBR) in the diet for two weeks prior to treatment (and/or during or after treatment) with a cancer inducing agent as described above.

Diets comprising 5% and 10% lyophilized black raspberries (LBR) prepared as described above and determine to comprise the following components as indicated above (Table 4) can be used.

The cancer agent can be applied to the oral cavities of the animals for eight weeks after which the animals were sacrificed 12-13 weeks from the beginning of treatment and the number and volume of tumors (mm³) can be determined and/or histological examination is conducted on tissue samples of the oral cavity. Significant differences in the number, volume, or incidence of tumors or the degree of tissue dysplasia determined by histological examination are evaluated in animals fed a berry extract as compared to control animals

Accordingly, the chemoprevention studies above, using a mixture of the tobacco-associated carcinogens, BaP and NNK, provide the ability to evaluate the anti-cancer properties of the berry extracts of the invention in a well-defined animal system that mimics the pathologic condition of former tobacco users.

### EXAMPLE 13

### Cell-Based Method Demonstrating the Anti-Colon Cancer Properties of Berry Extract Fractions

The following studies were performed to examine the anti-cancer properties of the berry extracts of the invention in human colon carcinoma.

In this example, the growth inhibitory effects of anthocyanin-rich black raspberry extracts on the growth of normal and cancerous human colon cell lines was examined. In particular, anthocyanin-rich extracts from black raspberries (Rubus occidentals) were investigated for their inhibitory properties on the proliferation of normal colon cell lines and cancerous colon cell lines (HT-29). All extracts (i.e., fractions DM, F001, F003, F004, and ET) inhibited the proliferation of the human colon cancer cell line, HT-29, within 24h of administration of the extract. Notably, colon cancer cells were more susceptible to growth inhibition by anthocyanin-rich extracts at concentrations of 5 to 50 μg/ml than normal human colon cells. Cell cycle analyses indicated that progression through the cell cycle was altered in extract-treated cells as compared to untreated controls.

These findings indicate that the anthocyanin-rich berry extracts of the invention can inhibit the growth of human colon cancer cells.

TABLE 8
Growth Inhibition of Human Colon Carcinoma Cells by Black
Raspberry Extract Fractions

| % Inhibition | | | Fraction |
|---|---|---|---|
| 24 h | 72 h | 6 d | in μg/ml |
| 0 | 0 | 0 | DM-0 |
| 42.23 | 9.031 | 6.843 | DM-05 |
| 42.72 | 24.215 | 2.87 | DM-25 |
| 45.63 | 33.639 | 7.947 | DM-50 |
| 0 | 0 | 0 | F001-0 |
| 3.07 | −0.386 | 33.483 | F001-05 |
| 16.667 | 42.6 | 30.562 | F001-25 |
| 40.789 | 37.58 | 35.506 | F001-50 |
| 0 | 0 | 0 | F003-0 |
| 13.0653 | 47.607 | 8.913 | F003-05 |
| 4.5226 | 50.453 | 18.004 | F003-25 |
| 3.518 | 48.124 | 24.777 | F003-50 |
| 0 | 0 | 0 | F004-0 |
| 37.433 | 12.87 | 29.448 | F004-05 |
| 47.594 | 16.86 | 40.286 | F004-25 |
| 44.92 | 19.949 | 37.014 | F004-50 |
| 0 | 0 | 0 | ET-0 |
| 32.738 | −6.7 | 29.4334 | ET-05 |
| 33.929 | 5.583 | 30.189 | ET-25 |
| 35.119 | 16.005 | 38.868 | ET-50 |

### EXAMPLE 14

### Cell-Based Method Demonstrating the Anti-Oral Cancer Properties of Berry Extract Fractions

The following studies were performed to examine the anti-oral cancer properties of the berry extracts of the invention in human oral carcinoma.

In particular, using a panel of normal, premalignant and malignant oral epithelial cell lines, the cellular (growth inhibiting and cytotoxic) effects of phytochemicals found in black raspberry fractions was determined (i.e., F001, F003, DM, and ME/Et, see FIGS. 1 and 2). The berry extract representing ˜55% of the total berry components (F001) did not affect the growth or induce cytotoxicity in the oral cell lines. However, partitioning and chromatography of the F001 extract yielded three fractions which exhibited varying degrees of growth inhibition in the oral cell lines. The water soluble F003 fraction exhibited no growth inhibiting effects to any the cell lines. The F001 berry fraction portioned into chloroform (F003) was selectively growth inhibitory to the premalignant oral cell line. Following silica gel column chromatography of the F001 extract, the fractions eluting with dichloromethane (DM) and methanol/ethanol (Me/Et) were selectively growth inhibitory to the premalignant and malignant cell lines. The extracts or fractions were not observed to be cytotoxic to the cells, indicating that phytochemicals in the DM, ethanol and methanol extracts are growth inhibitory without eliciting cytotoxicity. Coinciding with the selective growth inhibiting effects of DM and Et, the number of premalignant and malignant cells increased in the G₂/M phase of the cell cycle. Ellagic acid, a major component of berries, was tested and found to be a potent, non-selective, inhibitor of oral human cell growth.

These studies demonstrate that non-toxic doses of berry extracts, and components thereof, such as ellagic acid, are capable of inhibiting the proliferation of human oral precancerous and cancer cells.

### Equivalents

Those skilled in the art will recognize, or be able to ascertain using no more than routine experimentation, many equivalents to the specific embodiments of the invention described herein. Such equivalents are intended to be encompassed by the following claims.

The contents of all patents, patent applications, and references cited throughout this specification are hereby incorporated herein by reference in their entireties.

1. An isolated berry extract having a therapeutically effective amount of antioxidant activity.

2. The extract of claim 1, wherein the berry is selected from the group consisting of strawberry, raspberry, red raspberry, black raspberry, and a combination thereof.

3. The extract of claim 1, wherein the berry is a strawberry.

4. The extract of claim 1, wherein the berry is a black raspberry.

5. The extract of claim 1, wherein the antioxidant activity has an oxygen radical absorbance capacity (ORAC) value per mg. selected from the group consisting of at least about 5.0, 6.0, 7.0, 8.0, 9.0, 10.0, 11.0, 12.0, 13.0, 14.0, 15.0, 16.0, 17.0, 18.0, 19.0, and 20.0.

6. The extract of claim 1, comprising vitamin A.

7. The extract of claim 1, comprising a vitamin E (tocochromonal).

8. The extract of claim 1, comprising vitamin C (ascorbic acid).

9. The extract of claim 1, comprising folic acid.

10. The extract of claim 1, further comprising a carotenoid, a phenolic compound, a phytosterol, a mineral, or a combination thereof.

11. The extract of claim 10, wherein said carotenoid is selected from the group consisting of α-carotene, β-carotene, zeaxanthin, and lutein.

12. The extract of claim 10, wherein said phenolic compound is selected from the group consisting of ellagic acid, ferulic acid, anthocyanin, cyanidin, quercetin, pelargonidin, kaempferol, and analogs thereof.

13. The extract of claim 10, wherein said phytosterol is selected from the group consisting of β-sitosterol, campesterol, and stigmasterol, and analogs thereof.

14. The extract of claim 10, wherein said mineral is selected from the group consisting of calcium, magnesium, potassium, zinc, and selenium.

15. A foodstuff comprising an extract according to claim 1, or one or more components derived therefrom.

16. A dietary supplement comprising an extract according to claim 1, or one or more components derived therefrom.

17. A pharmaceutical composition comprising an extract according to claim 1, or one or more components derived therefrom, and a pharmaceutically acceptable carrier therefor.

18. A method for treating or preventing a disease or condition in a subject comprising the step of administering to said subject a therapeutically-effective amount of a foodstuff, dietary supplement or pharmaceutical composition of claims 15, 16 or 17, respectively.

19. The method of claim 18, wherein said disease or condition is selected from the group consisting of a malignancy, a cardiovascular disease, a thrombotic disease, an atherogenic disease, an inflammatory disease or condition, an immunological disease, a neurological disease, a dermatological disease, an opthalmological disease, or aging.

20. The method of claim 19, wherein the malignancy is an aerodigestive tract cancer.

20a. The method of claim 20, wherein said aerodigestive tract cancer is oral, esophageal, or colon cancer.

21. The method of claim 19, wherein said subject has, or is at risk for acquiring, a malignancy.

22. The method of claim 19, wherein said subject has or is at risk for acquiring a cardiovascular disease.

23. The method of claim 19, wherein said subject has, or is at risk for acquiring, a respiratory disease, an inflammatory disease or condition, an immunological disease or disorder, a neurological disease, a dermatological disease, an opthalmological disease, or a gastroenterological disease.

24. A method for isolating a berry extract comprising,
physically disrupting an amount of berries;
exposing the disrupted berries to low temperature; and
removing an amount of water content by sublimation.

25. The method of claim 24, further comprising the steps of
adding to the berry extract an organic solvent to produce an extract/solvent mixture; and
removing the solvent portion of the extract/solvent mixture thereby producing isolated berry extract fraction substantially free of solvent.

26. The method of claim 24, wherein the berries are selected from the group consisting of a strawberries, raspberries, red raspberries, black raspberries, and combinations thereof.

27. The method of claim 24, wherein the berries are strawberries.

28. The method of claim 24, wherein the berries are black raspberries.

29. The method of claim 24 or 25, wherein said removing is conducted under a vacuum.

30. The method of claim 29, wherein said vacuum is at least about 380 millitorr.

31. The method of claim 24 or 25, wherein the low temperature is at least about −20-C.

32. The method of claim 24, wherein the low temperature is between about −20-C and 20-C.

33. The method of claim 25, wherein said isolated berry extract is at least about 90% free of solvent.

34. The method of claim 25, wherein the organic solvent is selected from the group consisting of dichloromethane, methanol, ethanol, acetone, and combinations thereof.

35. The method of claim 34, wherein the organic solvent is about a 1:1 combination of dichloromethane and methanol.

36. The method of claim 34, wherein the organic solvent is about a 1:1 combination of dichloromethane and ethanol.

37. The method of claim 34, wherein the organic solvent is about a 1:1 combination of acetone and methanol.

38. The method of claim 34, wherein the organic solvent is about a 1:1 combination of acetone and ethanol.

39. The method of claim 25, wherein said extract fraction represents at least about 55% of the starting extract material.

40. An isolated berry extract produced by the method of claim 24.

41. An isolated berry extract fraction produced by the method of claim 25.

42. The isolated berry extract of claim 40, having a therapeutically effective amount of antioxidant activity.

43. The isolated berry extract of claim 40, comprising vitamin A.

44. The isolated berry extract of claim 40, comprising a vitamin E (tocochromanol).

45. The isolated berry extract of claim 40, comprising vitamin C (ascorbic acid).

46. The isolated berry extract of claim 40, comprising folic acid.

47. The isolated berry extract of claim 40, comprising a carotenoid, a phenolic compound, a phytosterol, a mineral, or a combination thereof.

48. The isolated berry extract of claim 47, wherein said carotenoid is selected from the group consisting of α-carotene, β-carotene, zeaxanthin, and lutein.

49. The isolated berry extract of claim 47, wherein said phenolic compound is selected from the group consisting of ellagic acid, ferulic acid, anthocyanins, cyanidin, quercetin, pelargonidin, kaempferol, and analogs thereof.

50. The isolated berry extract of claim 47, wherein said phytosterol is selected from the group consisting of β-sitosterol, campesterol, stigmasterol, and analogs thereof.

51. The isolated berry extract of claim 47, wherein said mineral is selected from the group consisting of calcium, magnesium, potassium, zinc, and selenium.

52. The isolated berry extract fraction of claim 41, comprising vitamin A.

53. The isolated berry extract fraction of claim 41, comprising a vitamin E (tochromanol).

54. The isolated berry extract fraction of claim 41, comprising vitamin C (ascorbic acid).

55. The isolated berry extract fraction of claim 41, comprising folic acid.

56. The isolated berry extract fraction of claim 41, comprising a carotenoid, a phenolic compound, a phytosterol, a mineral, or a combination thereof.

57. The isolated berry extract fraction of claim 41, wherein said carotenoid is selected from the group consisting of α-carotene, β-carotene, zeaxanthin, and lutein.

58. The isolated berry extract fraction of claim 41, wherein said phenolic compound is selected from the group consisting of ellagic acid, ferulic acid, anthocyanins, cyanidin, quercetin, pelargonidin, kaempferol, and analogs thereof.

59. The isolated berry extract fraction of claim 41, wherein said phytosterol is selected from the group consisting of β-sitosterol, campesterol, stigmasterol, and analogs thereof.

60. The isolated berry extract fraction of claim 41, wherein said mineral is selected from the group consisting of calcium, magnesium, potassium, zinc, and selenium.

61. The isolated berry extract of claim 40, in a form suitable for use in a foodstuff.

62. The isolated berry extract of claim 40, in a form suitable for use as a dietary supplement.

63. The isolated berry extract of claim 40, in a form suitable for use in a pharmaceutical composition.

64. The isolated berry extract fraction of claim 41, in a form suitable for use in a foodstuff.

65. The isolated berry extract fraction of claim 41, in a form suitable for use as a dietary supplement.

66. The isolated berry extract fraction of claim 41, in a form suitable for use in a pharmaceutical composition.

67. A foodstuff comprising the extract of claim 40 or extract fraction of claim 41.

68. A dietary supplement comprising the extract of claim 40 or extract fraction of claim 41.

69. A pharmaceutical composition comprising the extract of claim 40 or extract fraction of claim 41.

70. A method for treating or preventing a disease or condition in a subject comprising,
administering to said subject a therapeutically-effective amount of a composition comprising a berry extract or fraction thereof having antioxidant activity.

71. A method for treating or preventing a disease or condition in a subject comprising the step of administering to said subject a therapeutically-effective amount of a foodstuff, dietary supplement or pharmaceutical composition of claims 15, 16 or 17, respectively.

72. The method of claim 68, wherein said disease or condition is selected from the group consisting of a malignancy, a cardiovascular disease, a thrombotic disease, an atherogenic disease, an inflammatory disease or condition, an immunological disease or disorder, a neurological disease, a dermatological disease, an opthalmological disease, or aging.

73. The method of claim 72, wherein the malignancy is an aerodigestive tract cancer.

73a. The method of claims 72, wherein said aerodigestive tract cancer is oral, esophageal, or colon cancer.

74. The method of claim 71, wherein said subject has, or is at risk for acquiring, a malignancy.

75. The method of claim 71, wherein said subject has or is at risk for acquiring a cardiovascular disease.

76. A method of treating a subject in need of an antioxidant therapy comprising,
administering to said subject a therapeutically-effective amount of a composition comprising a berry extract or fraction thereof having antioxidant activity, such that antioxidant therapy is achieved.

77. The method of claim 71, wherein the subject in need of an antioxidant therapy has a disease or condition selected from the group consisting of a malignancy, a cardiovascular disease, a thrombotic disease, an atherogenic disease, an inflammatory disease, an immunological disease, a neurological disease, a dermatological disease, an opthalmological disease, or aging.

78. The method of claim 71, wherein the malignancy is an aerodigestive tract cancer.

78a. The method of claims 78, wherein said aerodigestive tract cancer is oral, esophageal, or colon cancer.

79. A method of treating an antioxidant responsive disease or condition comprising,
administering to a subject having said antioxidant responsive disease or condition a therapeutically-effective amount of a composition comprising a berry extract or fraction thereof having antioxidant activity, such that treatment is achieved.

80. The method of claim 79, wherein the antioxidant responsive condition is selected from the group consisting of a malignancy, a cardiovascular disease, a thrombotic disease, an atherogenic disease, an inflammatory disease or condition, an immunological disease or disorder, a neurological disease, a dermatological disease, an opthalmological disease, or aging.

81. The method of claim 80, wherein the malignancy is an aerodigestive tract cancer.

81a. The method of claims 81, wherein said aerodigestive tract cancer is oral, esophageal, or colon cancer.

82. The method of claim 80, wherein the malignancy is metastatic.

83. The method of claim 80, wherein the cardiovascular disease is hypercholesterolemia.

84. The method of any one of claims 70, 76, and 79, wherein the berry extract, or fraction thereof, is from a fruit selected from the group consisting of a strawberry, raspberry, red raspberry, black raspberry, and a combination thereof.

85. The method of any one of claims 70, 76, and 79, wherein the berry extract, or fraction thereof, is from a strawberry.

86. The method of any one of claims 70, 76, and 79, wherein the berry extract, or fraction thereof, is from a black raspberry.

87. The method of claim 84, wherein the composition further comprises a compound selected from group comprising a vitamin A, vitamin E (tocochromanol), vitamin C (ascorbic acid), folic acid, a carotenoid, a phenolic compound, a phytosterol, a mineral, or a combination thereof.

88. The method of claim 84, wherein the composition is administered orally.

89. A method of nutritionally supplementing a foodstuff comprising, adding to said foodstuff an isolated berry extract, or fraction thereof, from a berry selected from the group consisting of strawberry, raspberry, black raspberry, red raspberry, and a combination thereof.

90. A method of supplementing a dietary supplement comprising,
adding to said dietary supplement an isolated berry extract, or fraction thereof, from a berry selected from the group consisting of strawberry, raspberry, black raspberry, red raspberry, and a combination thereof.

91. A method of supplementing a pharmaceutical comprising,
adding to said pharmaceutical a composition selected from the group consisting of an isolated berry extract, or fraction thereof, from a berry selected from the group consisting of strawberry, raspberry, black raspberry, red raspberry and a combination thereof.

92. A composition comprising a compound isolated from a berry extract, or fraction thereof, and selected from the group consisting of an antioxidant, vitamin A, vitamin E (tocochromanol), vitamin C (ascorbic acid), folic acid, a carotenoid, a phenolic compound, a phytosterol, a mineral, or a combination thereof.

93. The composition of claim 92, wherein the berry extract, or fraction thereof, is from a berry selected from the group consisting of a strawberry, raspberry, black raspberry, red raspberry, and a combination thereof.

94. The composition of claim 93, wherein the berry is a strawberry.

95. The composition of claim 93, wherein the berry is a black raspberry.

96. The composition of claim 93, wherein said compound is present in an amount at least about 100% greater than present in the native berry.

97. The composition of claim 92, wherein said carotenoid is selected from the group consisting of α-carotene, β-carotene, zeaxanthin, and lutein.

98. The composition of claim 92, wherein said phenolic compound is selected from the group consisting of ellagic acid, ferulic acid, anthocyanins, cyanidin, quercetin, pelargonidin.

99. The composition of claim 92, wherein said phytosterol is selected from the group consisting of β-sitosterol, campesterol, stigmasterol, and analogs thereof.

100. The composition of claim 92, wherein said mineral is selected from the group consisting of calcium, magnesium, potassium, zinc, and selenium.

101. A foodstuff comprising a composition according to claim 92.

102. A dietary supplement comprising a composition according to claim 92.

103. A pharmaceutical comprising a composition according to claim 92.

104. A berry extract or fraction thereof of any one of claims 1, 40, or 41, suitable for modulating undesired signal transduction activity in a subject in need thereof.

105. A berry extract or fraction thereof of any one of claims 1, 40, or 41, suitable for modulating the metabolism of a carcinogen.

106. A berry extract or fraction thereof of any one of claims 1, 40, or 41, suitable for modulating the carcinogenic metabolite.

107. The extract or fraction of any one of claims 104, 105, and 106, wherein the extract has antioxidant activity.

108. The extract or fraction of claim 104, wherein the antioxidant activity has an oxygen radical absorbance capacity (ORAC) value per mg. selected from the group consisting of at least about 5.0, 6.0, 7.0, 8.0, 9.0, 10.0, 11.0, 12.0, 13.0, 14.0, 15.0, 16.0, 17.0, 18.0, 19.0, and 20.0.

109. The extract or fraction of any one of claims 104, 105, and 106, wherein the extract comprises vitamin A.

110. The extract or fraction of any one of claims 104, 105, and 106, wherein the extract comprises vitamin E (tocochromanol).

111. The extract or fraction of any one of claims 104, 105, and 106, wherein the extract comprises vitamin C (ascorbic acid).

112. The extract or fraction of any one of claims 104, 105, and 106, wherein the extract comprises folic acid.

113. The extract or fraction of any one of claims 104, 105, and 106, wherein the extract comprises a carotenoid, a phenolic compound, a phytosterol, a mineral, or a combination thereof.

114. The extract or fraction of any one of claims 1, 40, or 41, wherein a pharmaceutical is added to the extract or faction.
